(19) United States
(12) Patent Application Publication
(10) Pub. No.: US 2019/0135880 A1
(43) Pub. Date: May 9, 2019

(54) Modified Fibroin

(71) Applicants: Spiber Inc., Yamagata (JP); Kojima Industries Corporation, Aichi (JP)

(72) Inventors: Keisuke Morita, Yamagata (JP); Hiroyuki Nakamura, Yamagata (JP)

(73) Assignees: Spiber Inc., Yamagata (JP); Kojima Industries Corporation, Aichi (JP)

(21) Appl. No.: 16/096,818

(22) PCT Filed: Apr. 28, 2017
(86) PCT No.: PCT/JP2017/016925

§ 371 Date: Oct. 26, 2018

(30) Foreign Application Priority Data
Apr. 28, 2016 (JP) 2016-091196
Nov. 11, 2016 (JP) 2016-220744

(51) Int. Cl. C07K 14/435; C12N 15/86; C12N 15/70; C12N 15/74; C12N 15/81; D01F 4/02

(52) U.S. Cl. CPC C07K 14/43518; C12N 15/86; D01F 4/02; C12N 15/74; C12N 15/81; C12N 15/70

(57) ABSTRACT

Provided is a modified fibroin, including a domain sequence represented by Formula 1: [(A)ₙ motif-REP]ₘ, in which the domain sequence has an amino acid sequence having a reduced content of (A)ₙ motif equivalent to an amino acid sequence in which, at least, one or a plurality of the (A)ₙ motifs is deleted, as compared to naturally occurring fibroin.

[In Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, and a plurality of (A)ₙ motifs or the like may be the same amino acid sequence or different amino acid sequences.]

### TECHNICAL FIELD

The present invention relates to a modified fibroin. More specifically, the present invention relates to a modified fibroin having a reduced content of (A)ₙ motif. The present invention also relates to a nucleic acid encoding a modified fibroin, an expression vector including the nucleic acid sequence, a host transformed with the expression vector, and a product made from a modified fibroin.

### BACKGROUND ART

Fibroin is a type of fibrous protein and contains up to 90% of glycine, alanine and serine residues leading to the formation of a β-pleated sheet (Non-Patent Literature 1). Proteins (silk proteins, Hornet silk proteins, and spider silk proteins) and the like constituting the yarn produced by insects and spiders are known as fibroin.

Silk proteins have excellent mechanical properties, hygroscopic properties and deodorizing properties and are widely used as raw materials for garments. In addition, the silk yarn is an immuno-tolerant natural fiber and has high biocompatibility and is therefore also used for surgical sutures.

Up to seven types of silk glands exist in spider, each producing fibroin (spider silk protein) with different properties. According to the organ of the source, spider silk proteins are designated a major ampullate spider protein (MaSp) with high toughness, a minor ampullate spider protein (MiSp) with high elongation, and flagelliform (Flag), tubuliform, aggregate, acinifonii, and pyriform spider silk proteins. In particular, structural studies have been intensively conducted in the major ampullate spider protein exhibiting high toughness due to having excellent strength (stress and toughness) and elongation (Patent Literature 1 and 2).

As a structure specific to fibroin, a structure in which amino acid motifs classified as GPGXX, an extended region rich in alanine residues ((A)ₙ or (GA)ₙ), GGX, and a spacer are repeated is known (Non-Patent Literature 2). In addition, it has been reported that substitution of the (GA)ₙ motif with the (A)ₙ motif leads to decreased elongation but increased tensile strength, an increasing number of GPGXX motifs leads to increased elongation, and substitution of several GPGXX motifs with the (A)ₙ motifs leads to increased tensile strength (Patent Literature 2). In addition, the GGX and GPGXX motifs are thought to have a flexible helical structure that imparts elasticity to yarns (Patent Literature 3).

Recombinant spider silk proteins and recombinant silk proteins are produced in several heterologous protein production systems. For example, transgenic goat, transgenic silkworm, or recombinant plant or mammalian cells are utilized (Non-Patent Literature 3). However, these production systems exhibit a low production rate and are not suitable for mass production meeting the commercial level (Patent Literature 4 and Patent Literature 5). Although many cases of production of recombinant fibroin by organisms such as yeast, mold, gram-negative bacterium and gram-positive bacterium as a production system capable of mass production have also been reported and certain outcomes have been achieved, it has not been possible to achieve industrial mass production of the recombinant fibroin having excellent elongation and tensile strength (Patent Literature 5).

### CITATION LIST

### Patent Literature

[Patent Literature 1] Japanese Unexamined Patent Publication No. 2012-55269
[Patent Literature 2] Japanese Unexamined Patent Publication No. 2005-502347
[Patent Literature 3] Japanese Unexamined Patent Publication No. 2009-505668
[Patent Literature 4] Japanese Unexamined Patent Publication No. 2014-502140
[Patent Literature 5] International Patent Publication No. WO2015/042164

### Non Patent Literature

[Non-Patent Literature 1] Asakura et al., Encyclopedia of Agricultural Science, Academic Press: New York, N.Y., 1994, Vol. 4, pp. 1-11
[Non-Patent Literature 2] Microbial Cell Factories, 2004, 3:14
[Non-Patent Literature 3] Science, 2002, Vol. 295, pp. 472-476

### SUMMARY OF INVENTION

### Problems to be Solved by the Invention

Due to its excellent properties, fibroin has drawn attention as a new material in various industrial fields such as medicine, aviation, and clothing. However, it is necessary to further improve the productivity of fibroin in order to achieve an amount of production that meets the commercial level.

An object of the present invention is to provide a modified fibroin having improved productivity while maintaining the strength and elongation of fibroin.

### Means for Solving the Problems

As a result of various studies on methods capable of industrial mass production, the present inventors have unexpectedly found that the productivity of fibroin can be improved while maintaining stress, and toughness and elongation of fibroin can also be improved, by reducing the content of (A)ₙ motif, which is considered to be involved in strength (stress and toughness) of fibroin. The present invention is based on such novel findings.

That is, the present invention relates to, for example, each of the following inventions.

[1] A modified fibroin, including:

a domain sequence represented by Formula 1: [(A)ₙ motif-REP]ₘ,

in which the domain sequence has an amino acid sequence having a reduced content of (A)ₙ motif equivalent to an amino acid sequence in which, at least, one or a plurality of the (A)ₙ motifs is deleted, as compared to naturally occurring fibroin. [In Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.]

[2] The modified fibroin according to [1], in which the domain sequence has an amino acid sequence equivalent to an amino acid sequence in which, at least, one (A)ₙ motif per one to three (A)ₙ motifs from the N-terminal side to the C-terminal side is deleted, as compared to the naturally occurring fibroin.

[3] The modified fibroin according to [1], in which the domain sequence has an amino acid sequence equivalent to an amino acid sequence in which, at least, two consecutive (A)ₙ motif deletions and one (A)ₙ motif deletion are repeated in this order from the N-terminal side to the C-terminal side, as compared to the naturally occurring fibroin.

[4] A modified fibroin, including:

a domain sequence represented by Formula 1: [(A)ₙ motif-REP]ₘ,

in which x/y is 50% or more, in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙmotif-REP] units where the ratio of the number of amino acid residues in the other REP is 1.8 to 11.3 is defined as x, and the total number of amino acid residues of the domain sequence is defined as y.

[In Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ, motifs may be the same amino acid sequence or different amino acid sequences, and a plurality of REPs may be the same amino acid sequence or different amino acid sequences.]

[5] The modified fibroin according to any one of [1] to [4], in which the fibroin has, in addition to an amino acid sequence corresponding to deletion of one or a plurality of (A)ₙ motifs, an amino acid sequence corresponding to substitution, deletion, insertion and/or addition of one or a plurality of amino acid residues, as compared to naturally occurring fibroin.

[6] The modified fibroin according to [5], in which the naturally occurring fibroin is a fibroin derived from an insect or a spider. [7] The modified fibroin according to [5], in which the naturally occurring fibroin is a major ampullate spider protein (MaSp) or minor ampullate spider protein (MiSp) of spiders.

[8] The modified fibroin according to any one of [5] to [7], in which the domain sequence has an amino acid sequence equivalent to an amino acid sequence in which, at least, one (A)ₙ motif per one to three (A)ₙ motifs from the N-terminal side to the C-terminal side is deleted, as compared to the naturally occurring fibroin.

[9] The modified fibroin according to any one of [5] to [7], in which the domain sequence has an amino acid sequence equivalent to an amino acid sequence in which, at least, two consecutive (A)ₙ motif deletions and one (A)ₙ motif deletion are repeated in this order from the N-terminal side to the C-terminal side, as compared to the naturally occurring fibroin.

[10] The modified fibroin according to any one of [1] to [9], in which the domain sequence has an amino acid sequence having a reduced content of glycine residues equivalent to an amino acid sequence in which, at least, one or a plurality of the glycine residues in REP is substituted with another amino acid residue, as compared to the naturally occurring fibroin.

[11] The modified fibroin according to [10], in which the domain sequence has an amino acid sequence equivalent to an amino acid sequence in which, at least, in at least one motif sequence selected from GGX and GPGXX (where X represents an amino acid residue other than glycine) in REP, one glycine residue in one or a plurality of the motif sequences is substituted with another amino acid residue, as compared to the naturally occurring fibroin.

[12] The modified fibroin according to [11], in which the ratio of the motif sequence having the substitution of a glycine residue with another amino acid residue is 40% or more with respect to the entire motif sequence. [13] The modified fibroin according to any one of [10] to [12], in which z/w is 30% or more in the case where the total number of amino acid residues in the amino acid sequence consisting of XGX (where X represents an amino acid residue other than glycine) contained in all REPs in the sequence excluding the sequence from the (A)ₙ motif located at the most C-terminal side to the C-terminal of the domain sequence from the domain sequence is defined as z, and the total number of amino acid residues in the sequence excluding the sequence from the (A)ₙ motif located at the most C-terminal side to the C-terminal of the domain sequence from the domain sequence is defined as w.

[14] A modified fibroin, including an amino acid sequence set forth in SEQ ID NO: 2, SEQ ID NO: 4 or SEQ ID NO: 10, or an amino acid sequence having 90% or more sequence identity with the amino acid sequence set forth in SEQ ID NO: 2, SEQ ID NO: 4 or SEQ ID NO: 10.

[15] The modified fibroin according to any one of [1] to [14], further including a tag sequence at either or both of the N-terminal and the C-terminal.

[16] The modified fibroin according to [15], in which the tag sequence includes an amino acid sequence set forth in SEQ ID NO: 5.

[17] A modified fibroin, including an amino acid sequence set forth in SEQ ID NO: 7, SEQ ID NO: 9 or SEQ ID NO: 11, or an amino acid sequence having 90% or more sequence identity with the amino acid sequence set forth in SEQ ID NO: 7, SEQ ID NO: 9 or SEQ ID NO: 11.

[18] A nucleic acid encoding the modified fibroin according to any one of [1] to [17].

[19] A nucleic acid that hybridizes with a complementary strand of the nucleic acid according to [18] under stringent conditions and encodes a modified fibroin including a domain sequence represented by Formula 1: [(A)ₙ motif-REP]ₘ.

[In Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.]

[20] A nucleic acid having 90% or more sequence identity with the nucleic acid according to [18] and encoding a modified fibroin including a domain sequence represented by Formula 1: [(A)ₙ motif-REP]ₘ.

[In Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.]

[21] An expression vector, including the nucleic acid sequence according to any one of [18] to [20]; and one or a plurality of regulatory sequences operably linked thereto.

[22] The expression vector according to [21], which is a plasmid vector or a viral vector.

[23] A host transformed with the expression vector according to [21] or [22].

[24] The host according to [23], which is a prokaryote.

[25] The host according to [24], in which the prokaryote is a microorganism belonging to a genus selected from the group consisting of Escherichia, Brevibacillus, Serratia, Bacillus, Microbacterium, Brevibacterium, Corynebacterium and Pseudomonas.

[26] The host according to [23], which is a eukaryote.

[27] The host according to [26], in which the eukaryote is a yeast, a filamentous fungus or an insect cell.

[28] The host according to [27], in which the yeast is a yeast belonging to a genus selected from the group consisting of Saccharomyces, Schizosaccharomyces, Kluyveromyces, Trichosporon, Schwanniomyces, Pichia, Candida, Yarrowia and Hansenula.

[29] The host according to [28], in which the yeast belonging to the genus Saccharomyces is Saccharomyces cerevisiae, the yeast belonging to the genus Schizosaccharomyces is Schizosaccharomyces pombe, the yeast belonging to the genus Kluyveromyces is Kluyveromyces lactis, the yeast belonging to the genus Trichosporon is Trichosporon pullulans, the yeast belonging to the genus Schwaniomyces is Schwanniomyces alluvius, the yeast belonging to the genus Pichia is Pichia pastoris, the yeast belonging to the genus Candida is Candida albicans, the yeast belonging to the genus Yarrowia is Yarrowia lipolytica, and the yeast belonging to the genus Hansenula is Hansenula polymorpha.

[30] The host according to [27], in which the filamentous fungus is a filamentous fungus belonging to a genus selected from the group consisting of Aspergillus, Penicillium and Mucor.

[31] The host according to [30], in which the filamentous fungus belonging to the genus Aspergillus is Aspergillus oryzae, the filamentous fungus belonging to the genus Penicillium is Penicillium chrysogenum, and the filamentous fungus belonging to the genus Mucor is Mucor fragilis.

[32] The host according to [27], in which the insect cell is a lepidopteran insect cell.

[33] The host according to [27], in which the insect cell is an insect cell derived from Spodoptera frugiperda or an insect cell derived from Trichoplusia ni.

[34] A product including the modified fibroin according to any one of [1] to [17] and selected from the group consisting of a fiber, a yarn, a filament, a film, a foam, a sphere, a nanofibril, a hydrogel, a resin and an equivalent thereof.

### Effects of the Invention

According to the present invention, it is possible to provide a modified fibroin having improved productivity while maintaining the strength and elongation of fibroin. Since the (A)ₙ motif of fibroin have been considered to be closely related to the strength (stress and toughness) of fibroin, research and development have been advanced to increase the content of the (A)ₙ motif so far, and it has been thought that the strength of fibroin is significantly decreased by decreasing the content of the (A)ₙ motif. However, the present inventors have found that, even in the case where the content of the (A)ₙ motif is decreased, the stress of fibroin does not decrease significantly, the amount of production of fibroin in the recombinant protein production system can be significantly improved, and further the toughness and elongation of fibroin are also improved. According to the present invention, such an unexpected effect is also exerted.

### BRIEF DESCRIPTION OF DRAWINGS

FIG. 1 is a schematic diagram showing a domain sequence of a modified fibroin.

FIG. 2 is a diagram showing a distribution of values of x/y (%) of naturally occurring fibroin.

FIG. 3 is a diagram showing a distribution of values of z/w (%) of naturally occurring fibroin.

### EMBODIMENTS FOR CARRYING OUT THE INVENTION

Hereinafter, embodiments for carrying out the present invention will be described in detail. However, the present invention is not limited to the following embodiments.

[Modified Fibroin]

The modified fibroin according to the present invention is a protein including a domain sequence represented by Formula 1: [(A)ₙ motif-REP]ₘ. In the modified fibroin, an amino acid sequence (N-terminal sequence and C-terminal sequence) may be further added to either or both of the N-terminal side and the C-terminal side of the domain sequence. The N-terminal sequence and the C-terminal sequence, although not limited thereto, are typically regions that do not have repetitions of amino acid motifs characteristic of fibroin and consist of amino acids of about 100 residues.

The term “modified fibroin” as used herein means a fibroin whose domain sequence is different from the amino acid sequence of naturally occurring fibroin. The term “naturally occurring fibroin” as used herein is also a protein including a domain sequence represented by Formula 1: [(A)ₙ motif-REP]ₘ.

The “modified fibroin” may be a fibroin whose amino acid sequence has been modified based on naturally occurring fibroin (for example, a fibroin whose amino acid sequence has been modified by altering a gene sequence of cloned naturally occurring fibroin) or a fibroin artificially designed and synthesized independently of naturally occurring fibroin (for example, a fibroin having a desired amino acid sequence by chemically synthesizing a nucleic acid encoding the designed amino acid sequence), as long as it has the amino acid sequence specified in the present invention.

The term “domain sequence” as used herein refers to an amino acid sequence which produces a crystalline region (which typically corresponds to (A)ₙ motif of an amino acid sequence) and an amorphous region (which typically corresponds to REP of an amino acid sequence) peculiar to fibroin and means an amino acid sequence represented by Formula 1: [(A)ₙ motif-REP]ₘ. Here, the (A)ₙ motif represents an amino acid sequence consisting of 4 to 20 amino acid residues and the number of alanine residues relative to the total number of amino acid residues in the (A)ₙ motif is 83% or more. The REP represents an amino acid sequence consisting of 10 to 200 amino acid residues. m represents an integer of 8 to 300. A plurality of (A)ₙ motifs may be the same amino acid sequence or different amino acid sequences. A plurality of REPS may be the same amino acid sequence or different amino acid sequences.

The (A)ₙ motif may be such that the number of alanine residues is 83% or more relative to the total number of amino acid residues in the (A)ₙ motif, preferably 86% or more, more preferably 90% or more, still more preferably 95% or more, and even still more preferably 100% (which means that the (A)ₙ motif consists of only alanine residues). It is preferred that at least seven of a plurality of (A)ₙ motifs in the domain sequence consist of only alanine residues. The phrase “consist of only alanine residues” means that the (A)ₙ motif has an amino acid sequence represented by (A)ₙ (where A represents an alanine residue and n represents an integer of 4 to 20 and preferably an integer of 4 to 16).

The modified fibroin according to one embodiment has an amino acid sequence whose domain sequence has a reduced content of the (A)ₙ motif as compared to naturally occurring fibroin. The domain sequence of the modified fibroin can be said to have an amino acid sequence equivalent to an amino acid sequence in which, at least, one or a plurality of the (A)ₙ motifs is deleted, as compared to naturally occurring fibroin.

The modified fibroin according to the present embodiment may be, for example, a modified fibroin having an amino acid sequence equivalent to an amino acid sequence in which 10 to 40% of the (A)ₙ motif is deleted from naturally occurring fibroin. In the case where the decrease in the content of the (A)ₙ motif is within this range, it is possible to more stably exert the effect of the present invention that the amount of production of fibroin in the recombinant protein production system can be significantly improved without significantly reducing the strength thereof (stress and toughness).

In the modified fibroin according to the present embodiment, the domain sequence preferably has an amino acid sequence equivalent to an amino acid sequence in which, at least, one (A)ₙ motif per one to three (A)ₙ motifs from the N-terminal side to the C-terminal side is deleted, as compared to naturally occurring fibroin. By this configuration, the effect of the present invention is more significantly exhibited.

In the modified fibroin according to the present embodiment, the domain sequence preferably has an amino acid sequence equivalent to an amino acid sequence in which, at least, two consecutive (A)ₙ motif deletions and one (A)ₙ motif deletion are repeated in this order from the N-terminal side to the C-terminal side, as compared to the naturally occurring fibroin. By this configuration, the effect of the present invention is more significantly exhibited.

In the modified fibroin according to the present embodiment, the domain sequence preferably has an amino acid sequence equivalent to an amino acid sequence in which, at least, (A)ₙ motif every other two positions is deleted from the N-terminal side to the C-terminal side. By this configuration, the effect of the present invention is more significantly exhibited.

The modified fibroin according to the present embodiment may further have modifications of an amino acid sequence corresponding to substitution, deletion, insertion and/or addition of one or a plurality of amino acid residues as compared to naturally occurring fibroin, in addition to the modification on the (A)ₙ motif described above.

The modified fibroin according to the present embodiment can be obtained, for example, from a gene sequence of cloned naturally occurring fibroin, by deleting one or a plurality of the sequences encoding the (A)ₙ motif. Further, for example, the modified fibroin according to the present embodiment can also be obtained by designing an amino acid sequence corresponding to deletion of one or a plurality of the (A)ₙ motifs, from the amino acid sequence of naturally occurring fibroin, and chemically synthesizing a nucleic acid encoding the designed amino acid sequence. In any case, in addition to the modification corresponding to deletion of the (A)ₙ motif from the amino acid sequence of naturally occurring fibroin, further modification of the amino acid sequence corresponding to substitution, deletion, insertion and/or addition of one or a plurality of amino acid residues may be carried out. Substitution, deletion, insertion and/or addition of amino acid residues can be carried out by methods well known to those skilled in the art, such as site-directed mutagenesis. Specifically, it can be carried out according to a method described in literatures such as Nucleic Acid Res. 10, 6487 (1982), and Methods in Enzymology, 100, 448 (1983).

Naturally occurring fibroin is a protein including a domain sequence represented by Formula 1: [(A)ₙ motif-REP]ₘ, specifically, for example, a fibroin produced by insects or spiders.

Examples of the fibroin produced by insects include silk proteins produced by silkworms such as Bombyx mori, Bombyx mandarina, Antheraea yamamai, Anteraea pernyi, Eriogyna pyretorum, Pilosamia Cynthia ricini, Sarnia cynthia, Caligura japonica, Antheraea mylitta, and Antheraea assama; and Hornet silk proteins discharged by larvae of Vespa simillima xanthoptera.

A more specific example of the fibroin produced by insects may be a silkworm fibroin L chain (GenBank Accession No. M76430 (nucleotide sequence), AAA27840.1 (amino acid sequence)).

Examples of the fibroin produced by spiders include spider silk proteins produced by spiders belonging to the genus Araneus such as Araneus ventricosus, Araneus diadematus, Araneus pinguis, Araneus pentagrammicus and Araneus nojimai, spiders belonging to the genus Neoscona such as Neoscona scylla, Neoscona nautica, Neoscona adianta and Neoscona scylloides, spiders belonging to the genus Pronus such as Pronous minutes, spiders belonging to the genus Cyrtarachne such as Cyrtarachne bufo and Cyrtarachne inaequalis, spiders belonging to the genus Gasteracantha such as Gasteracantha kuhli and Gasteracantha mammosa, spiders belonging to the genus Ordgarius such as Ordgarius hobsoni and Ordgarius sexspinosus, spiders belonging to the genus Argiope such as Argiope amoena, Argiope minuta and Argiope bruennich, spiders belonging to the genus Arachnura such as Arachnura logia, spiders belonging to the genus Acusilas such as Acusilas coccineus, spiders belonging to the genus Cytophora such as Cyrtophora moluccensis, Cyrtophora exanthematica and Cyrtophora unicolor, spiders belonging to the genus Poltys such as Poltys illepidus, spiders belonging to the genus Cyclosa such as Cyclosa octotuberculata, Cyclosa sedeculata, Cyclosa vallata and Cyclosa atrata, and spiders belonging to the genus Chorizopes such as Chorizopes nipponicus; and spider silk proteins produced by spiders belonging to the genus Tetragnatha such as Tetragnatha praedonia, Tetragnatha maxillosa, Tetragnatha extensa and Tetragnatha squamata, spiders belonging to the genus Leucauge such as Leucauge magnifica, Leucauge blanda and Leucauge subblanda, spiders belonging to the genus Nephila such as Nephila clavata and Nephila pilipes, spiders belonging to the genus Menosira such as Menosira ornata, spiders belonging to the genus Dyschiriognatha such as Dyschiriognatha tenera, spiders belonging to the genus Latrodectus such as Latrodectus mactans, Latrodectus hasseltii, Latrodectus geometricus and Latrodectus tredecimguttatus, and spiders belonging to the family Tetragnathidae such as spiders belonging to the genus Euprosthenops. Examples of spider silk proteins include traction fiber proteins such as MaSp (MaSp1 and MaSp2) and ADF (ADF3 and ADF4), and MiSp (MiSp1 and MiSp2).

More specific examples of the fibroin produced by spiders include fibroin-3 (adf-3) [derived from Araneus diadematus] (GenBank Accession Number AAC47010 (amino acid sequence), U47855 (nucleotide sequence)), fibroin-4 (adf-4) [derived from Araneus diadematus] (GenBank Accession Number AAC47011 (amino acid sequence), U47856 (nucleotide sequence)), dragline silk protein spidroin 1 [derived from Nephila clavipes] (GenBank Accession Number AAC04504 (amino acid sequence), U37520 (nucleotide sequence)), major angullate spidroin 1 [derived from Latrodectus hesperus] (GenBank Accession Number ABR68856 (amino acid sequence)), EF595246 (nucleotide sequence)), dragline silk protein spidroin 2 [derived from Nephila clavata] (GenBank Accession Number AAL32472 (amino acid sequence), AF441245 (nucleotide sequence)), major anpullate spidroin 1 [derived from Euprosthenops australis] (GenBank Accession Number CAJ00428 (amino acid sequence), AJ973155 (nucleotide sequence)) and major ampullate spidroin 2 [Euprosthenops australis] (GenBank Accession Number CAM32249.1 (amino acid sequence), AM490169 (nucleotide sequence)), minor ampullate silk protein 1 [Nephila clavipes] (GenBank Accession Number AAC14589.1 (amino acid sequence)), minor ampullate silk protein 2 [Nephila clavipes] (GenBank Accession Number AAC14591.1 (amino acid sequence)), and minor ampullate spidroin-like protein [Nephilengys cruentata] (GenBank Accession Number ABR37278.1 (amino acid sequence).

As a more specific example of naturally occurring fibroin, fibroin in which sequence information is registered in NCBI GenBank can be further mentioned. For example, it can be confirmed by extracting sequences in which spidroin, ampullate, fibroin, “silk and polypeptide”, or “silk and protein” is described as a keyword in DEFINITION among sequences containing INV as DIVISION among sequence information registered in NCBI GenBank, sequences in which a specific character string of products is described from CDS, or sequences in which a specific character string is described from SOURCE to TISSUE TYPE.

The modified fibroin according to another embodiment includes a domain sequence represented by Formula 1: [(A)ₙ motif-REP]ₘ, and has an amino acid sequence in which x/y is 50% or more, in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙ motif-REP] units where the ratio of the number of amino acid residues in the other REP is 1.8 to 11.3 is defined as x, and the total number of amino acid residues of the domain sequence is defined as y. Since the content of the (A)ₙ motif in the modified fibroin according to the present embodiment is reduced, the ratio at which the ratio of the number of amino acid residues in REP of two adjacent [(A)ₙ motif-REP] units falls within the above-specified range is high.

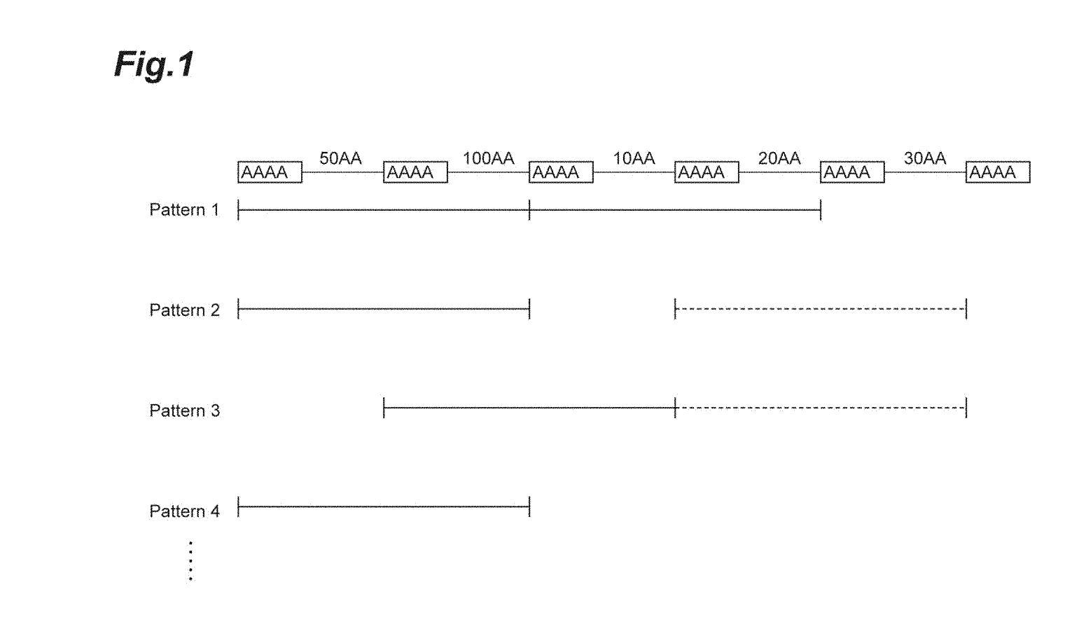

A method of calculating x/y will be described in more detail with reference to FIG. 1. FIG. 1 shows a domain sequence excluding N-terminal sequence and C-terminal sequence from modified fibroin. This domain sequence has a sequence of (A)ₙ motif-first REP (50 amino acid residues)-(A)ₙ motif-second REP (100 amino acid residues)-(A)ₙ motif-third REP (10 amino acid residues)-(A)ₙ motif-fourth REP (20 amino acid residues)-(A)ₙ motif-fifth REP (30 amino acid residues)-(A)ₙ motif from the N-terminal side (left side).

The two adjacent [(A)ₙ motif-REP] units are sequentially selected from the N-terminal side to the C-terminal side so as not to overlap. At this time, an unselected [(A)ₙ motif-REP] unit may exist. FIG. 1 shows a pattern 1 (a comparison between first REP and second REP and a comparison between third REP and fourth REP), a pattern 2 (a comparison between first REP and second REP and a comparison between fourth REP and fifth REP), a pattern 3 (a comparison between second REP and third REP and a comparison between fourth REP and fifth REP), and a pattern 4 (a comparison between first REP and second REP). There are other selection methods besides this.

Next, for each pattern, the number of amino acid residues of each REP in the selected two adjacent [(A)ₙ motif-REP] units is compared. The comparison is carried out by obtaining the ratio of the number of amino acid residues of the other REP in the case where one REP having a smaller number of amino acid residues is 1. For example, in the case of comparing the first REP (50 amino acid residues) and the second REP (100 amino acid residues), the ratio of the number of amino acid residues of the second REP is 100/50=2 in the case where the first REP having a smaller number of amino acid residues is 1. Similarly, in the case of comparing the fourth REP (20 amino acid residues) and the fifth REP (30 amino acid residues), the ratio of the number of amino acid residues of the fifth REP is 30/20=1.5 in the case where the fourth REP having a smaller number of amino acid residues is 1.

In FIG. 1, a set of [(A)ₙ motif-REP] units in which the ratio of the number of amino acid residues of the other REP is 1.8 to 11.3 in the case where one REP having a smaller number of amino acid residues is 1 is indicated by a solid line. Hereinafter, such a ratio is referred to as a Giza ratio. A set of [(A)ₙ motif-REP] units in which the ratio of the number of amino acid residues of the other REP is less than 1.8 or more than 11.3 in the case where one REP having a smaller number of amino acid residues is 1 is indicated by a broken line.

In each pattern, the number of all amino acid residues of two adjacent [(A)ₙ motif-REP] units indicated by solid lines (including not only the number of amino acid residues of REP but also the number of amino acid residues of (A)ₙ motif) is combined. Then, the total values thus combined are compared and the total value of the pattern whose total value is the maximum (the maximum value of the total value) is defined as x. In the example shown in FIG. 1, the total value of the pattern 1 is the maximum.

Next, x/y (%) can be calculated by dividing x by the total amino acid residue number y of the domain sequence.

In the modified fibroin according to the present embodiment, x/y is preferably 50% or more, more preferably 60% or more, still more preferably 65% or more, even still more preferably 70% or more, even further preferably 75% or more, and particularly preferably 80% or more. The upper limit of x/y is not particularly limited, and may be, for example, 100% or less. In the case where the Giza ratio is 1:1.9 to 11.3, x/y is preferably 89.6% or more; in the case where the Giza ratio is 1:1.8 to 3.4, x/y is preferably 77.1% or more; in the case where the Giza ratio is 1:1.9 to 8.4, x/y is preferably 75.9% or more; and in the case where the Giza ratio is 1:1.9 to 4.1, x/y is preferably 64.2% or more.

In the case where the modified fibroin according to the present embodiment is a modified fibroin in which at least seven of a plurality of the (A)ₙ motifs in the domain sequence consist of only alanine residues, x/y is preferably 46.4% or more, more preferably 50% or more, still more preferably 55% or more, even still more preferably 60% or more, still further preferably 70% or more, and particularly preferably 80% or more. The upper limit of x/y is not particularly limited, and it may be 100% or less.

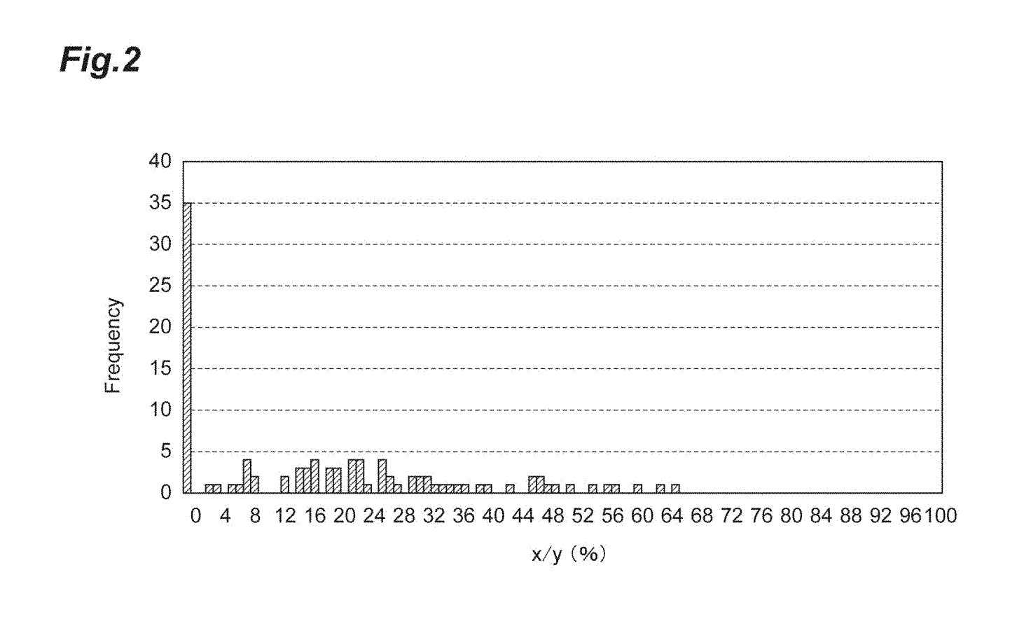

Here, x/y in naturally occurring fibroin will be described. First, as described above, 663 types of fibroins (415 types of fibroins derived from spiders among them) were extracted by confirming fibroins with amino acid sequence information registered in NCBI GenBank by a method exemplified. x/y was calculated by the above-mentioned calculation method from the amino acid sequences of naturally occurring fibroins consisting of a domain sequence represented by Formula 1: [(A)ₙ motif-REP]ₘ, among all the extracted fibroins. FIG. 2 shows the results in the case where the Giza ratio is 1:1.9 to 4.1.

In FIG. 2, the horizontal axis represents x/y (%) and the vertical axis represents frequency. As is clear from FIG. 2, x/y in naturally occurring fibroin is less than 64.2% (highest, 64.14%).

The modified fibroin according to the present embodiment can be obtained, for example, from a gene sequence of cloned naturally occurring fibroin, by deleting one or a plurality of the sequences encoding the (A)ₙ motif such that x/y is 64.2% or more. Further, the modified fibroin according to the present embodiment can also be obtained, for example, by designing an amino acid sequence corresponding to deletion of one or a plurality of (A)ₙ motifs such that x/y is 64.2% or more, from the amino acid sequence of naturally occurring fibroin, and chemically synthesizing a nucleic acid encoding the designed amino acid sequence. In any case, in addition to the modification corresponding to deletion of the (A)ₙ motif from the amino acid sequence of naturally occurring fibroin, further modification of the amino acid sequence corresponding to substitution, deletion, insertion and/or addition of one or a plurality of amino acid residues may be carried out.

In the modified fibroin of the present invention, it is preferred that the domain sequence has an amino acid sequence in which the content of glycine residues is reduced in addition to having a reduced content of the (A)ₙ motif as compared to naturally occurring fibroin. By this configuration, the effect of the present invention is more significantly exhibited. The domain sequence of the modified fibroin can be said to have an amino acid sequence equivalent to an amino acid sequence in which one or a plurality of glycine residues in REP is substituted with another amino acid residue, as well as at least one or a plurality of (A)ₙ motifs is deleted, as compared to naturally occurring fibroin.

Next, a specific embodiment of the domain sequence in which the content of glycine residues is reduced will be described. Although the description on the reduction of the content of (A)ₙ motif is omitted, each of the above embodiments relating to the reduction of the content of (A)ₙ motif and each of following embodiments relating to the reduction of the content of glycine residues can be arbitrarily combined.

In the modified fibroin according to one embodiment, the domain sequence has an amino acid sequence equivalent to an amino acid sequence in which, at least, in at least one motif sequence selected from GGX and GPGXX (where X represents an amino acid residue other than glycine) in REP, one glycine residue in one or a plurality of the motif sequences is substituted with another amino acid residue, as compared to the naturally occurring fibroin.

Since the GGX motif and the GPGXX motif of fibroin were considered to be involved in the elongation of the fibroin fiber, substitution of the glycine residue (G) of these motifs with another amino acid residue was thought to greatly affect the elongation of this fibroin. However, the present inventors have found that substitution of one G in the GGX motif and GPGXX motif with another amino acid does not affect the elongation of the fibroin fiber by leaving the other G remaining, and additionally the amount of production of fibroin in the recombinant protein production system can be significantly improved. According to the modified fibroin of the present embodiment, such an unexpected effect is exerted.

In the present embodiment, it is preferred that the ratio of the motif sequence having the substitution of a glycine residue with another amino acid residue is 40% or more with respect to the entire motif sequence. By this configuration, the above-mentioned effects can be more significantly exhibited.

The modified fibroin according to another embodiment has an amino acid sequence in which z/w is 30% or more in the case where the total number of amino acid residues in the amino acid sequence consisting of XGX (where X represents an amino acid residue other than glycine) contained in all REPs in the sequence excluding the sequence from the (A)ₙ motif located at the most C-terminal side to the C-terminal of the domain sequence from the domain sequence is defined as z, and the total number of amino acid residues in the sequence excluding the sequence from the (A)ₙ motif located at the most C-terminal side to the C-terminal of the domain sequence from the domain sequence is defined as w.

The calculation method of z/w will be described in more detail. First, an amino acid sequence consisting of XGX is extracted from all the REPs in the sequence excluding the sequence from the (A)ₙ motif located at the most C-terminal side to the C-terminal of the domain sequence from the domain sequence. The total number of amino acid residues constituting XGX is z. For example, in the case where 50 amino acid sequences consisting of XGX are extracted (there is no overlap), z is 50×3=150. Also, for example, in the case where X (central X) contained in two XGXs exists as in the case of the amino acid sequence consisting of XGXGX, it is calculated by subtracting the overlapping portion (in the case of XGXGX, it is 5 amino acid residues). w is the total number of amino acid residues contained in the sequence excluding the sequence from the (A)ₙ motif located at the most C-terminal side to the C-terminal of the domain sequence from the domain sequence. For example, in the case of the domain sequence shown in FIG. 1, w is 4+50+4+100+4+10+4+20+4+30=230 (excluding the (A)ₙ motif located at the most C-terminal side). Next, z/w (%) can be calculated by dividing z by w. z/w corresponds to the content ratio of the amino acid sequence consisting of XGX.

In the modified fibroin according to the present embodiment, it is preferable to increase the content ratio of the amino acid sequence consisting of XGX by substituting one glycine residue of the GGX motif with another amino acid residue. In the modified fibroin according to the present embodiment, the content ratio of the amino acid sequence consisting of GGX in the domain sequence is preferably 6% or less, more preferably 4% or less, and still more preferably 2% or less. The content ratio of the amino acid sequence consisting of GGX in the domain sequence can be calculated by the same method as the calculation method of the content ratio (z/w) of the amino acid sequence consisting of XGX described above.

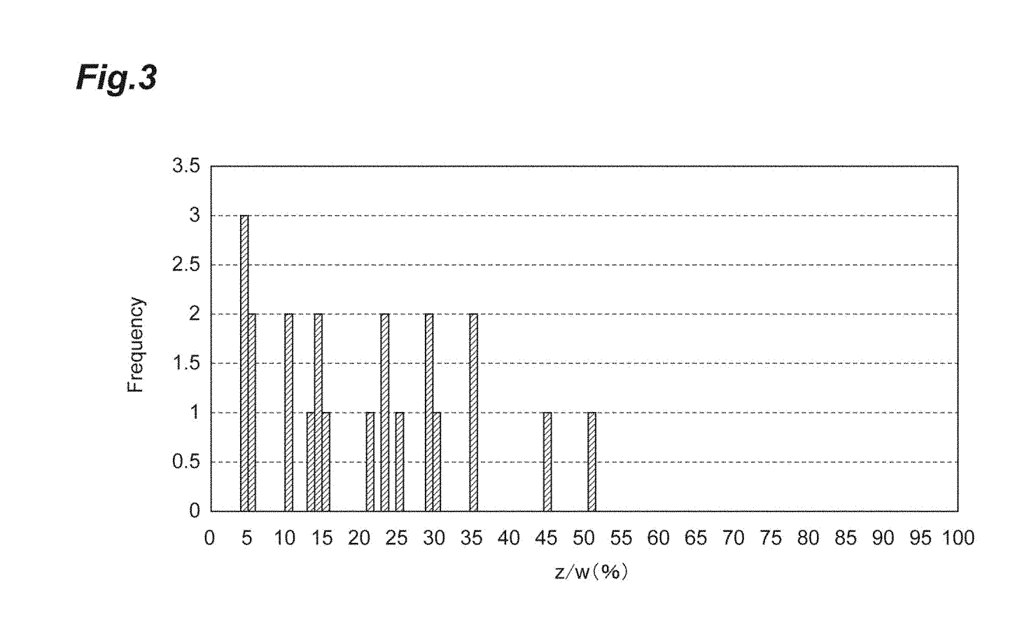

Here, z/w in naturally occurring fibroin will be described. First, as described above, 663 types of fibroins (415 types of fibroins derived from spiders among them) were extracted by confirming fibroins with amino acid sequence information registered in NCBI GenBank by a method exemplified. z/w was calculated by the above-mentioned calculation method from the amino acid sequences of naturally occurring fibroins which include a domain sequence represented by Formula 1: [(A)ₙ motif-REP]ₘ and in which the content ratio of the amino acid sequence consisting of GGX in the fibroin is 6% or less, among all the extracted fibroins. The results are shown in FIG. 3. In FIG. 3, the horizontal axis represents z/w (%) and the vertical axis represents frequency. z/w in naturally occurring fibroin is less than 50.9% (highest, 50.86%). In the naturally occurring fibroin which includes a domain sequence represented by Formula 1: [(A)ₙ motif-REP]ₘ and in which at least seven of a plurality of the (A)ₙ motifs in the domain sequence consist of only alanine residues (as described above, x/y is less than 46.4%), the effect is recognized in the case where z/w is 14.2% or more.

In the modified fibroin according to the present embodiment, z/w is preferably 30% or more, more preferably 50% or more, still more preferably 50.9% or more, even still more preferably 56.2% or more, still further preferably 70% or more, and particularly preferably 75% or more. The upper limit of z/w is not particularly limited, but it may be 95% or less, for example.

The modified fibroin including a domain sequence with a reduced content of glycine residues can be obtained, for example, by substituting and modifying at least a part of a nucleotide sequence encoding a glycine residue from the gene sequence of cloned naturally occurring fibroin so as to encode another amino acid residue. At this time, one glycine residue in the GGX motif and GPGXX motif may be selected as the glycine residue to be modified, and substitution may be carried out such that z/w is equal to or more than the above-mentioned value. Alternatively, the modified fibroin can also be obtained, for example, by designing an amino acid sequence satisfying each of the above embodiments from the amino acid sequence of naturally occurring fibroin and chemically synthesizing a nucleic acid encoding the designed amino acid sequence.

In the case of substituting and modifying at least a part of the nucleotide sequence encoding a glycine residue so as to encode another amino acid residue, the another amino acid residue is not particularly limited as long as it is an amino acid residue other than a glycine residue, but it is preferably a hydrophobic amino acid residue such as a valine (V) residue, a leucine (L) residue, an isoleucine (I) residue, a methionine (M) residue, a proline (P) residue, a phenylalanine (F) residue, or a tryptophan (W) residue, or a hydrophilic amino acid residue such as a glutamine (Q) residue, an asparagine (N) residue, a serine (S) residue, a lysine (K) residue, or a glutamic acid (E) residue, among which more preferred is a valine (V) residue, a leucine (L) residue, an isoleucine (I) residue or a glutamine (Q) residue, and still more preferred is a glutamine (Q) residue.

A more specific example of the modified fibroin according to the present invention may be a modified fibroin including (i) an amino acid sequence set forth in SEQ ID NO: 2, SEQ ID NO: 4 or SEQ ID NO: 10, or (ii) an amino acid sequence having 90% or more sequence identity with the amino acid sequence set forth in SEQ ID NO: 2, SEQ ID NO: 4 or SEQ ID NO: 10.

The modified fibroin of (i) will be described. The amino acid sequence set forth in SEQ ID NO: 2 is the amino acid sequence in which (A)ₙ motif every other two positions from the N-terminal side to the C-terminal side from the amino acid sequence set forth in SEQ ID NO: 1, which corresponds to naturally occurring fibroin, is deleted and further one [(A)ₙ motif-REP] is inserted before the C-terminal sequence. The amino acid sequence set forth in SEQ ID NO: 4 is the amino acid sequence in which all GGX in REP of the amino acid sequence set forth in SEQ ID NO: 2 is substituted with GQX. The amino acid sequence set forth in SEQ ID NO: 10 is the amino acid sequence in which two alanine residues are inserted at the C-terminal side of each (A)ₙ motif of the amino acid sequence set forth in SEQ ID NO: 4 and further a part of glutamine (Q) residues is substituted with a serine (S) residue and a part of amino acids on the N-terminal side is deleted so as to be almost the same as the molecular weight of SEQ ID NO: 4. In addition, the amino acid sequence set forth in SEQ ID NO: 3 is the amino acid sequence in which all GGX in REP of the amino acid sequence set forth in SEQ ID NO: 1 is substituted with GQX.

The value of x/y at Giza ratio 1:1.8 to 11.3 of the amino acid sequence set forth in SEQ ID NO: 1 (corresponding to naturally occurring fibroin) is 15.0% (see Table 1). The values of x/y in the amino acid sequence set forth in SEQ ID NO: 2 and the amino acid sequence set forth in SEQ ID NO: 4 are all 93.4% (see Tables 2 and 3). The value of x/y in the amino acid sequence set forth in SEQ ID NO: 10 is 92.7% (see Table 4). The values of z/w in the amino acid sequences set forth in SEQ ID NOs: 1, 2, 4 and 10 are 46.8%, 56.2%, 70.1% and 66.1%, respectively (see Table 5).

TABLE 1

| | Number of | Number of | | | | | | |
|---|---|---|---|---|---|---|---|---|
| Met-PRT313 | residues | residues | | | | | | |
| (SEQ ID NO: 1) | in unit | in REP | Pattern 1 | | Pattern 2 | | Pattern 3 | |
| | | | | Total | | Total | | Total |
| | | | | number | | number | | number |
| | | | Ratio of | of residues | Ratio of | of residues | Ratio of | of residues |
| | | | number of | in two | number of | in two | number of | in two |
| | | | residues | units | residues | units | residues | units |
| MGPGGQGPYGPG | — | — | — | — | — | — | — | — |
| (N-terminal | | | | | | | | |
| sequence) | | | | | | | | |
| ASAAAAAGGNGPGS | 22 | 15 | 1.2 | — | 1.2 | — | 1.4 | — |
| GQQGPGGS | | | | | | | | |
| AAAAAGGYGPGGQG | 23 | 18 | 1.3 | — | — | — | 1.8 | 42 |
| PGQQGPGSS | | | | | | | | |
| AAAAAGPGGYGPGG | 18 | 13 | 1.2 | — | 1.8 | 42 | 1.0 | — |
| QGPS | | | | | | | | |
| ASAAAAAGPGSGQQ | 17 | 10 | 1.2 | — | 1.0 | — | 1.0 | — |
| GPG | | | | | | | | |
| ASAAAAAGGYGPGG | 25 | 18 | 1.4 | — | 1.0 | — | 1.4 | — |
| QGPGQQGPGSS | | | | | | | | |
| AAAAAGGYGSGPGQ | 20 | 15 | 1.0 | — | 1.4 | — | 1.2 | — |
| QGPYGS | | | | | | | | |
| AAAAAGPGSGGYGQ | 20 | 15 | 1.3 | — | 1.2 | — | 1.8 | 43 |
| GPYGPG | | | | | | | | |
| ASAAAAAGPGGYGP | 20 | 13 | 1.6 | — | 1.8 | 43 | 1.4 | — |
| GGQGPS | | | | | | | | |
| ASAAAAAGSGQQGP | 20 | 13 | 1.2 | — | 1.4 | — | 1.0 | — |
| GGYGPY | | | | | | | | |
| ASAAAAAGGYGSGP | 25 | 18 | 1.5 | — | 1.0 | — | 1.2 | — |
| GQQGPYGPGGS | | | | | | | | |
| AAAAAGSGQQGPGQ | 18 | 13 | 1.4 | — | 1.2 | — | 1.2 | — |
| QGPY | | | | | | | | |
| ASAAAAAGPGGQGP | 20 | 13 | 1.1 | — | 1.2 | — | 1.6 | — |
| YGPGSS | | | | | | | | |
| AAAAAGGYGYGPGG | 21 | 16 | 2.0 | — | 1.6 | — | 1.3 | — |
| QGPYGPG | | | | | | | | |
| ASAAAAAGGNGPGS | 27 | 20 | — | — | 1.3 | — | — | — |
| GGYGPGQQGPGGS | | | | | | | | |
| AAAAAGPGGQGPYG | 16 | 11 | | | — | — | | |
| PG | | | | | | | | |
| ASAAAAAGGYGPGG | 25 | 18 | | | | | | |
| QGPGGYGPGSS | | | | | | | | |
| AAAAAGPGGQGPYG | 18 | 13 | | | | | | |
| PGSS | | | | | | | | |
| AAAAAGGYGPGQQG | 21 | 16 | | | | | | |
| PYGPGGS | | | | | | | | |
| AAAAAGGYQQGPGG | 21 | 16 | | | | | | |
| QGPYGPG | | | | | | | | |
| ASAAAAAGPGGQGP | 18 | 11 | | | | | | |
| YGPG | | | | | | | | |
| ASAAAAAGPGGYGP | 20 | 13 | | | | | | |
| GGQGPS | | | | | | | | |
| ASAAAAAGGYGSGA | 25 | 18 | | | | | | |
| GGYGPYGPGGS | | | | | | | | |
| AAAAAGPGSGQQGQ | 20 | 15 | | | | | | |
| GPYGPG | | | | | | | | |
| ASAAAAAGGYGPGQ | 23 | 16 | | | | | | |
| QGPYGPGGS | | | | | | | | |
| AAAAAGPGSGGYGP | 15 | 10 | | | | | | |
| G | | | | | | | | |
| ASAAAAAGGNGPGS | 27 | 20 | | | | | | |
| GGYGPGQQGPGGS | | | | | | | | |
| AAAAAGGYQQGPGG | 21 | 16 | | | | | | |
| QGPYGPG | | | | | | | | |
| ASAAAAAGPGSGQQ | — | — | | | | | | |
| GPGAS | | | | | | | | |
| (C-terminal | | | | | | | | |
| sequence) | | | | | | | | |
| | | | Total | | Total | | Total | |
| | | | number | | number | | number | |
| | | | of residues | | of residues | | of residues | |
| | | | at ratio of | | at ratio of | | at ratio of | |
| | | | 1.8 to 11.3 | | 1.8 to 11.3 | | 1.8 to 11.3 | |
| | | | (x1) | 42 | (x2) | 85 | (x3) | 85 |
| Total number | 566 | | x1/y = | 7.4% | x2/y = | 15.0% | x3/y = | 15.0% |
| of amino acid | | | | | | | | |
| residues in | | | | | | | | |
| domain | | | | | | | | |
| sequence | | | | | | | | |
| (y) = | | | | | | | | |

TABLE 2

| | Number of | Number of | | | | | | |
|---|---|---|---|---|---|---|---|---|
| Met-PRT399 | residues | residues | | | | | | |
| (SEQ ID NO: 2) | in unit | in REP | Pattern 1 | | Pattern 2 | | Pattern 3 | |
| | | | | Total | | Total | | Total |
| | | | | number | | number | | number |
| | | | Ratio of | of residues | Ratio of | of residues | Ratio of | of residues |
| | | | number of | in two | number of | in two | number of | in two |
| | | | residues | units | residues | units | residues | units |
| MGPGGQGPYGPG | — | — | — | — | — | — | — | — |
| (N-terminal | | | | | | | | |
| sequence) | | | | | | | | |
| ASAAAAAGGNGPGS | 40 | 33 | 2.5 | 58 | 2.5 | 58 | 2.3 | 55 |
| GQQGPGGSGGYGPG | | | | | | | | |
| GQGPGQQGPGSS | | | | | | | | |
| AAAAAGPGGYGPGG | 18 | 13 | 2.0 | 57 | — | — | 2.0 | 55 |
| QGPS | | | | | | | | |
| ASAAAAAGPGSGQQ | 37 | 30 | 2.3 | 55 | 2.0 | 55 | 2.4 | 58 |
| GPGASGGYGPGGQG | | | | | | | | |
| PGQQGPGSS | | | | | | | | |
| AAAAAGGYGSGPGQ | 20 | 15 | 2.4 | 58 | 2.4 | 58 | 2.9 | 63 |
| QOPYGS | | | | | | | | |
| AAAAAGPGSGGYGQ | 35 | 30 | 3.5 | 59 | 2.9 | 63 | 2.8 | 54 |
| GPYGPGASGPGGYG | | | | | | | | |
| PGGQGPS | | | | | | | | |
| ASAAAAAGSGQQGP | 20 | 13 | 1.9 | 59 | 2.8 | 54 | 1.8 | 55 |
| GGYGPY | | | | | | | | |
| ASAAAAAGGYGSGP | 38 | 31 | 2.2 | 54 | 1.8 | 55 | 2.5 | 60 |
| GQQGPYGPGGSGSG | | | | | | | | |
| QQGPGQQGPY | | | | | | | | |
| ASAAAAAGPGGQGP | 20 | 13 | 2.1 | 63 | 2.5 | 60 | 2.0 | 60 |
| YGPGSS | | | | | | | | |
| AAAAAGGYGYGPGG | 43 | 38 | 2.0 | 58 | 2.0 | 60 | 1.9 | 59 |
| QGPYGPGASGGNGP | | | | | | | | |
| GSGGYGPGQQGPGG | | | | | | | | |
| S | | | | | | | | |
| AAAAAGPGGQGPYG | 16 | 11 | — | — | 1.9 | 59 | — | — |
| PG | | | | | | | | |
| ASAAAAAGGYGPGG | 38 | 31 | | | — | — | | |
| QGPGGYGPGSSGPG | | | | | | | | |
| GQGPYGPGSS | | | | | | | | |
| AAAAAGGYGPGQQG | 21 | 16 | | | | | | |
| PYGPGGS | | | | | | | | |
| AAAAAGGYQQGPGG | 34 | 29 | | | | | | |
| QGPYGPGASGPGGQ | | | | | | | | |
| GPYGPG | | | | | | | | |
| ASAAAAAGPGGYGP | 20 | 13 | | | | | | |
| GGQGPS | | | | | | | | |
| ASAAAAAGGYGSGP | 40 | 33 | | | | | | |
| GGYGPYGPGGSGPG | | | | | | | | |
| SGQQGQGPYGPG | | | | | | | | |
| ASAAAAAGGYGPGQ | 23 | 16 | | | | | | |
| QGPYGPGGS | | | | | | | | |
| AAAAAGPGSGGYGP | 37 | 32 | | | | | | |
| GASGGNGPGSGGYG | | | | | | | | |
| PGQQGPGGS | | | | | | | | |
| AAAAAGGYQQGPGG | 21 | 16 | | | | | | |
| QGPYGPG | | | | | | | | |
| ASAAAAAGGYGSGP | 38 | 31 | | | | | | |
| GQQGPYGPGGSGSG | | | | | | | | |
| QQGPGQQOPY | | | | | | | | |
| ASAAAAAGPGSGQQ | — | — | | | | | | |
| GPGAS | | | | | | | | |
| (C-terminal | | | | | | | | |
| sequence) | | | | | | | | |
| | | | Total | | Total | | Total | |
| | | | number | | number | | number | |
| | | | of residues | | of residues | | of residues | |
| | | | at ratio of | | at ratio of | | at ratio of | |
| | | | 1.8 to 11.3 | | 1.8 to 11.3 | | 1.8 to 11.3 | |
| | | | (x1) | 521 | (x2) | 522 | (x3) | 519 |
| Total number | 559 | | x1/y = | 93.2% | x2/y = | 93.4% | x3/y = | 92.8% |
| of amino acid | | | | | | | | |
| residues in | | | | | | | | |
| domain | | | | | | | | |
| sequence | | | | | | | | |
| (y) = | | | | | | | | |

TABLE 3

| | Number of | Number of | | | | | | |
|---|---|---|---|---|---|---|---|---|
| Met-PRT410 | residues | residues | | | | | | |
| (SEQ ID NO: 4) | in unit | in REP | Pattern 1 | | Pattern 2 | | Pattern 3 | |
| | | | | Total | | Total | | Total |
| | | | | number | | number | | number |
| | | | Ratio of | of residues | Ratio of | of residues | Ratio of | of residues |
| | | | number of | in two | number of | in two | number of | in two |
| | | | residues | units | residues | units | residues | units |
| MGPGQQGPYGPG | — | — | — | — | — | — | — | — |
| (N-terminal | | | | | | | | |
| sequence) | | | | | | | | |
| ASAAAAAGQNGPGS | 40 | 33 | 2.5 | 58 | 2.5 | 58 | 2.3 | 55 |
| GQQGPGQSGQYGPG | | | | | | | | |
| QQGPGQQGPGSS | | | | | | | | |
| AAAAAGPGQYGPGQ | 18 | 13 | 2.0 | 57 | — | — | 2.0 | 55 |
| QGPS | | | | | | | | |
| ASAAAAAGPGSGQQ | 37 | 30 | 2.3 | 55 | 2.0 | 55 | 2.4 | 58 |
| GPGASGQYGPGQQG | | | | | | | | |
| PGQQGPGSS | | | | | | | | |
| AAAAAGQYGSGPGQ | 20 | 15 | 2.4 | 58 | 2.4 | 58 | 2.9 | 63 |
| QGPYGS | | | | | | | | |
| AAAAAGPGSGQYGQ | 35 | 30 | 3.5 | 59 | 2.9 | 63 | 2.8 | 54 |
| GPYGPGASGPGQYG | | | | | | | | |
| PGQQGPS | | | | | | | | |
| ASAAAAAGSGQQGP | 20 | 13 | 1.9 | 59 | 2.8 | 54 | 1.8 | 55 |
| GQYGPY | | | | | | | | |
| ASAAAAAGQYGSGP | 38 | 31 | 2.2 | 54 | 1.8 | 55 | 2.5 | 60 |
| GQQGPYGPGQSGSG | | | | | | | | |
| QQGPGQQGPY | | | | | | | | |
| ASAAAAAGPGQQGP | 20 | 13 | 2.1 | 63 | 2.5 | 60 | 2.0 | 60 |
| YGPGSS | | | | | | | | |
| AAAAAGQYGYGPGQ | 43 | 38 | 2.0 | 58 | 2.0 | 60 | 1.9 | 59 |
| QGPYGPGASGQNGP | | | | | | | | |
| GSGQYGPGQQGPGQ | | | | | | | | |
| S | | | | | | | | |
| AAAAAGPGQQGPYG | 16 | 11 | — | — | 1.9 | 59 | — | — |
| PG | | | | | | | | |
| ASAAAAAGQYGPGQ | 38 | 31 | | | — | — | | |
| QGPGQYGPGSSGPG | | | | | | | | |
| QQGPYGPGSS | | | | | | | | |
| AAAAAGQYGPGQQG | 21 | 16 | | | | | | |
| PYGPGQS | | | | | | | | |
| AAAAAGQYQQGPGQ | 34 | 29 | | | | | | |
| QGPYGPGASGPGQQ | | | | | | | | |
| GPYGPG | | | | | | | | |
| ASAAAAAGPGQYGP | 20 | 13 | | | | | | |
| GQQGPS | | | | | | | | |
| ASAAAAAGQYGSGP | 40 | 33 | | | | | | |
| GQYGPYGPGQSGPG | | | | | | | | |
| SGQQGQGPYGPG | | | | | | | | |
| ASAAAAAGQYGPGQ | 23 | 16 | | | | | | |
| QGPYGPGQS | | | | | | | | |
| AAAAAGPGSGQYGP | 37 | 32 | | | | | | |
| GASGQNGPGSGQYG | | | | | | | | |
| PGQQGPGQS | | | | | | | | |
| AAAAAGQYQQGPGQ | 21 | 16 | | | | | | |
| QGPYGPG | | | | | | | | |
| ASAAAAAGQYGSGP | 38 | 31 | | | | | | |
| GQQGPYGPGQSGSG | | | | | | | | |
| QQGPGQQGPY | | | | | | | | |
| ASAAAAAGPGSGQQ | — | — | | | | | | |
| GPGAS | | | | | | | | |
| (C-terminal | | | | | | | | |
| sequence) | | | | | | | | |
| | | | Total | | Total | | Total | |
| | | | number | | number | | number | |
| | | | of residues | | of residues | | of residues | |
| | | | at ratio of | | at ratio of | | at ratio of | |
| | | | 1.8 to 11.3 | | 1.8 to 11.3 | | 1.8 to 11.3 | |
| | | | (x1) | 521 | (x2) | 522 | (x3) | 519 |
| Total number | 559 | | x1/y = | 93.2% | x2/y = | 93.4% | x3/y = | 92.8% |
| of amino acid | | | | | | | | |
| residues in | | | | | | | | |
| domain | | | | | | | | |
| sequence | | | | | | | | |
| (y) = | | | | | | | | |

TABLE 4

| Met-PRT468 | Number of | Number of | | | | | | |
|---|---|---|---|---|---|---|---|---|
| (SEQ ID | residues | residues | | | | | | |
| NO: 10) | in unit | in REP | Pattern 1 | | Pattern 2 | | Pattern 3 | |
| | | | | Total | | Total | | Total |
| | | | | number | | number | | number |
| | | | Ratio of | of residues | Ratio of | of residues | Ratio of | of residues |
| | | | number of | in two | number of | in two | number of | in two |
| | | | residues | units | residues | units | residues | units |
| MGPGQQGPYGPG | — | — | — | — | — | — | — | — |
| (N-terminal | | | | | | | | |
| sequence) | | | | | | | | |
| ASAAAAAAAGSNGP | 42 | 33 | 2.5 | 62 | 2.5 | 62 | 2.3 | 59 |
| GSGQQGPGQSGQYG | | | | | | | | |
| PGQQGPGQQGPGSS | | | | | | | | |
| AAAAAAAGPGQYGP | 20 | 13 | 2.0 | 61 | — | — | 2.0 | 59 |
| GQQGPS | | | | | | | | |
| ASAAAAAAAGPGSG | 39 | 30 | 2.3 | 59 | 2.0 | 59 | 2.4 | 62 |
| QQGPGASGQYGPGQ | | | | | | | | |
| QGPGQQGPGSS | | | | | | | | |
| AAAAAAAGSYGSGP | 22 | 15 | 2.4 | 62 | 2.4 | 62 | 2.9 | 67 |
| GQQGPYGS | | | | | | | | |
| AAAAAAAGPGSGQY | 37 | 30 | 3.5 | 63 | 2.9 | 67 | 2.8 | 58 |
| GQGPYGPGASGPGQ | | | | | | | | |
| YGPGQQGPS | | | | | | | | |
| ASAAAAAAAGSGQQ | 22 | 13 | 1.9 | 63 | 2.8 | 58 | 1.8 | 59 |
| GPGQYGPY | | | | | | | | |
| ASAAAAAAAGSYGS | 40 | 31 | 2.2 | 58 | 1.8 | 59 | 2.5 | 64 |
| GPGQQGPYGPGQSG | | | | | | | | |
| SGQQGPGQQGPY | | | | | | | | |
| ASAAAAAAAGPGQQ | 22 | 13 | 2.1 | 67 | 2.5 | 64 | 2.0 | 64 |
| GPYGPGSS | | | | | | | | |
| AAAAAAAGSYGYGP | 45 | 38 | — | — | 2.0 | 64 | — | — |
| GQQGPYGPGASGQN | | | | | | | | |
| GPGSGQYGPGQQGP | | | | | | | | |
| GPS | | | | | | | | |
| AAAAAAAGPGQQGP | 18 | 11 | | | — | — | | |
| YGPG | | | | | | | | |
| ASAAAAAAAGSYGP | 40 | 31 | | | | | | |
| GQQGPGQYGPGSSG | | | | | | | | |
| PGQQGPYGPGSS | | | | | | | | |
| AAAAAAAGSYGPGQ | 23 | 16 | | | | | | |
| QGPYGPGPS | | | | | | | | |
| AAAAAAAGSYQQGP | 36 | 29 | | | | | | |
| GQQGPYGPGASGPG | | | | | | | | |
| QQGPYGPG | | | | | | | | |
| ASAAAAAAAGPGQY | 22 | 13 | | | | | | |
| GPGQQGPS | | | | | | | | |
| ASAAAAAAAGSYGS | 42 | 33 | | | | | | |
| GPGQYGPYGPGQSG | | | | | | | | |
| PGSGQQGQGPYGPG | | | | | | | | |
| ASAAAAAAAGSYGP | 25 | 16 | | | | | | |
| GQGGPYGPGPS | | | | | | | | |
| AAAAAAAGPGSGQY | 39 | 32 | | | | | | |
| QPGASGQNGPGSGQ | | | | | | | | |
| YGPGQQGPGPS | | | | | | | | |
| AAAAAAAGPGSGQQ | — | — | | | | | | |
| GPGAS | | | | | | | | |
| (C-terminal | | | | | | | | |
| sequence) | | | | | | | | |
| | | | Total | | Total | | Total | |
| | | | number | | number | | number | |
| | | | of residues | | of residues | | of residues | |
| | | | at ratio of | | at ratio of | | at ratio of | |
| | | | 1.8 to 11.3 | | 1.8 to 11.3 | | 1.8 to 11.3 | |
| | | | (x1) | 495 | (x2) | 495 | (x3) | 492 |
| Total number | 534 | | x1/y = | 92.7% | x2/y = | 92.7% | x3/y = | 92.1% |
| of amino acid | | | | | | | | |
| residues in | | | | | | | | |
| domain | | | | | | | | |
| sequence | | | | | | | | |
| (y) = | | | | | | | | |

TABLE 5

| | | | Total number | | |
|---|---|---|---|---|---|
| | Num- | | (z) of amino | Total value | |
| | ber | Over- | acid residues | (w) of amino | |
| | of | lapping | constituting | acid residues | z/w |
| Origin | XGX | AA | XGX | in domain | (%) |
| Met-PRT313 | 97 | 26 | 265 | 566 | 46.8 |
| (SEQ ID NO: 1) | | | | | |
| Met-PRT399 | 117 | 37 | 314 | 559 | 56.2 |
| (SEQ ID NO: 2) | | | | | |
| Met-PRT410 | 152 | 64 | 392 | 559 | 70.1 |
| (SEQ ID NO: 4) | | | | | |
| Met-PRT468 | 137 | 58 | 353 | 534 | 66.1 |
| (SEQ ID NO: 10) | | | | | |

The modified fibroin of (i) may consist of the amino acid sequence set forth in SEQ ID NO: 2, SEQ ID NO: 4 or SEQ ID NO: 10.

The modified fibroin of (ii) includes an amino acid sequence having 90% or more sequence identity with the amino acid sequence set forth in SEQ ID NO: 2, SEQ ID NO: 4 or SEQ ID NO: 10. The modified fibroin of (ii) is also a protein including a domain sequence represented by Formula 1: [(A)ₙ motif-REP]ₘ. The sequence identity is preferably 95% or more.

It is preferred that the modified fibroin of (ii) has 90% or more sequence identity with the amino acid sequence set forth in SEQ ID NO: 2, SEQ ID NO: 4 or SEQ ID NO: 10, and x/y is 64.2% or more in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙ motif-REP] units where the ratio of the number of amino acid residues in the other REP is 1.8 to 11.3 (Giza ratio is 1:1.8 to 11.3) is defined as x, and the total number of amino acid residues of the domain sequence is defined as y.

The above-mentioned modified fibroin may include a tag sequence at either or both of the N-terminal and C-terminal. This makes it possible to isolate, immobilize, detect and visualize the modified fibroin.

The tag sequence may be, for example, an affinity tag utilizing specific affinity (binding property, affinity) with another molecule. As a specific example of the affinity tag, a histidine tag (His tag) can be mentioned. The His tag is a short peptide in which about 4 to 10 histidine residues are arranged and has a property of specifically binding to a metal ion such as nickel, so it can be used for isolation of modified fibroin by chelating metal chromatography. A specific example of the tag sequence may be an amino acid sequence set forth in SEQ ID NO: 5 (amino acid sequence including His tag).

In addition, a tag sequence such as glutathione-S-transferase (GST) that specifically binds to glutathione or a maltose binding protein (MBP) that specifically binds to maltose can also be used.

Further, an “epitope tag” utilizing an antigen-antibody reaction can also be used. By adding a peptide (epitope) showing antigenicity as a tag sequence, an antibody against the epitope can be bound. Examples of the epitope tag include an HA (peptide sequence of hemagglutinin of influenza virus) tag, a myc tag, and a FLAG tag. The modified fibroin can easily be purified with high specificity by utilizing an epitope tag.

It is also possible to use a tag sequence which can be cleaved with a specific protease. By treating a protein adsorbed through the tag sequence with protease, it is also possible to recover the modified fibroin cleaved from the tag sequence.

A more specific example of the modified fibroin including a tag sequence may be a modified fibroin including (iii) an amino acid sequence set forth in SEQ ID NO: 7, SEQ ID NO: 9 or SEQ ID NO: 11, or (iv) an amino acid sequence having 90% or more sequence identity with the amino acid sequence set forth in SEQ ID NO: 7, SEQ ID NO: 9 or SEQ ID NO: 11.

The amino acid sequences set forth in SEQ ID NOs: 6, 7, 8, 9 and 11 are amino acid sequences in which an amino acid sequence set forth in SEQ ID NO: 5 (including a His tag) is added at the N-terminals of the amino acid sequences set forth in SEQ ID NOs: 1, 2, 3, 4 and 10, respectively.

The modified fibroin of (iii) may consist of an amino acid sequence set forth in SEQ ID NO: 7, SEQ ID NO: 9 or SEQ ID NO: 11.

The modified fibroin of (iv) includes an amino acid sequence having 90% or more sequence identity with the amino acid sequence set forth in SEQ ID NO: 7, SEQ ID NO: 9 or SEQ ID NO: 11. The modified fibroin of (iv) is also a protein including a domain sequence represented by Formula 1: [(A)ₙ motif-REP]ₘ. The sequence identity is preferably 95% or more.

It is preferred that the modified fibroin of (iv) has 90% or more sequence identity with the amino acid sequence set forth in SEQ ID NO: 7, SEQ ID NO: 9 or SEQ ID NO: 11, and x/y is 64.2% or more in the case where the number of amino acid residues in REPS of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙ motif-REP] units where the ratio of the number of amino acid residues in the other REP is 1.8 to 11.3 is defined as x, and the total number of amino acid residues of the domain sequence is defined as y.

The above-mentioned modified fibroin may include a secretory signal for releasing the protein produced in the recombinant protein production system to the outside of a host. The sequence of the secretory signal can be appropriately set depending on the type of the host.

[Nucleic Acid]

The nucleic acid according to the present invention encodes the modified fibroin according to the present invention. Specific examples of the nucleic acid include nucleic acids encoding a modified fibroin including an amino acid sequence set forth in SEQ ID NO: 2 or SEQ ID NO: 4, or a protein having an amino acid sequence (tag sequence) set forth in SEQ ID NO: 5 attached to either or both of the N-terminal and C-terminal of these amino acid sequences or the like.

The nucleic acid according to one embodiment is a nucleic acid which hybridizes with a complementary strand of a nucleic acid encoding the modified fibroin according to the present invention under stringent conditions and which encodes a modified fibroin including a domain sequence represented by Formula 1: [(A)ₙ motif-REP], in which x/y is 64.2% or more in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙ motif-REP] units where the ratio of the number of amino acid residues in the other REP is 1.8 to 11.3 is defined as x, and the total number of amino acid residues of the domain sequence is defined as y.

The term “stringent conditions” refers to conditions under which a so-called specific hybrid is formed and a non-specific hybrid is not formed. The “stringent conditions” may be any of low stringent conditions, moderately stringent conditions and highly stringent conditions. The low stringent conditions mean that hybridization occurs only in the case where there is at least 85% or more identity between the sequences, and include, for example, conditions of hybridization at 42° C. using 5×SSC containing 0.5% SDS. The moderately stringent conditions mean that hybridization occurs only in the case where there is at least 90% or more identity between the sequences, and include, for example, conditions of hybridization at 50° C. using 5×SSC containing 0.5% SDS. The highly stringent conditions mean that hybridization occurs only in the case where there is at least 95% or more identity between the sequences, and include, for example, conditions of hybridization at 60° C. using 5×SSC containing 0.5% SDS.

The nucleic acid according to one embodiment is a nucleic acid which has 90% or more sequence identity with a nucleic acid encoding the modified fibroin according to the present invention and which encodes a modified fibroin including a domain sequence represented by Formula 1: [(A)ₙ motif-REP]ₘ, in which x/y is 64.2% or more in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙ-REP] units where the ratio of the number of amino acid residues in the other REP is 1.8 to 11.3 is defined as x, and the total number of amino acid residues of the domain sequence is defined as y. The sequence identity is preferably 95% or more.

[Host and Expression Vector]

An expression vector according to the present invention has a nucleic acid sequence according to the present invention and one or a plurality of regulatory sequences operably linked thereto. The regulatory sequence is a sequence (for example, a promoter, an enhancer, a ribosome binding sequence, or a transcription termination sequence) that controls the expression of a recombinant protein in a host, and can be appropriately selected depending on the type of the host. The type of the expression vector such as a plasmid vector, a viral vector, a cosmid vector, a fosmid vector, or an artificial chromosome vector can be appropriately selected depending on the type of the host.

The host according to the present invention is a host which has been transformed with the expression vector according to the present invention. Both prokaryotes and eukaryotes such as yeast, filamentous fungi, insect cells, animal cells, and plant cells can be suitably used as hosts.

As the expression vector, an expression vector which can autonomously replicate in a host cell or can be incorporated into a chromosome of a host and which contains a promoter at a position capable of transcribing the nucleic acid according to the present invention is suitably used.

In the case where a prokaryote such as a bacterium is used as a host, the expression vector according to the present invention is preferably a vector which is capable of autonomous replication in the prokaryote and at the same time includes a promoter, a ribosome binding sequence, a nucleic acid according to the present invention and a transcription termination sequence. A gene that controls a promoter may be included.

Examples of the prokaryote include microorganisms belonging to the genus Escherichia, Brevibacillus, Serratia, Bacillus, Microbacterium, Brevibacterium, Corynebacterium and Pseudomonas.

Examples of microorganisms belonging to the genus Escherichia include Escherichia coli BL21 (Novagen, Inc.), Escherichia coli BL21 (DE3) (Life Technologies Corporation), Escherichia coli BLR (DE3) (Merck KGaA), Escherichia coli DH1, Escherichia coli GI698, Escherichia coli HB101, Escherichia coli JM109, Escherichia coli K5 (ATCC 23506), Escherichia coli KY3276, Escherichia coli MC1000, Escherichia coli MG1655 (ATCC 47076), Escherichia coli No. 49, Escherichia coli Rosetta (DE3) (Novagen, Inc.), Escherichia coli TB1, Escherichia coli Tuner (Novagen, Inc.), Escherichia coli Tuner (DE3) (Novagen, Inc.), Escherichia coli W1485, Escherichia coli W3110 (ATCC 27325), Escherichia coli XL1-Blue, and Escherichia coli XL2-Blue.

Examples of microorganisms belonging to the genus Brevibacillus include Brevibacillus agri, Brevibacillus borstelensis, Brevibacillus centrosporus, Brevibacillus formosus, Brevibacillus invocatus, Brevibacillus laterosporus, Brevibacillus limnophilus, Brevibacillus parabrevis, Brevibacillus reuszeri, Brevibacillus thermoruber, Brevibacillus brevis 47 (FERM BP-1223), Brevibacillus brevis 47K (FERM BP-2308), Brevibacillus brevis 47-5 (FERM BP-1664), Brevibacillus brevis 47-5Q (JCM 8975), Brevibacillus choshinensis HPD31 (FERM BP-1087), Brevibacillus choshinensis HPD31-S (FERM BP-6623), Brevibacillus choshinensis HPD31-OK (FERM BP-4573), and Brevibacillus choshinensis SP3 strain (manufactured by Takara Bio, Inc.).

Examples of microorganisms belonging to the genus Serratia include Serratia liquefaciens ATCC 14460, Serratia entomophila, Serratia ficaria, Serratia fonticola, Serratia grimesii, Serratia proteamaculans, Serratia odorifera, Serratia plymuthica, and Serratia rubidaea.

Examples of microorganisms belonging to the genus Bacillus include Bacillus subtilis and Bacillus amyloliquefaciens.

Examples of microorganisms belonging to the genus Microbacterium include Microbacterium ammoniaphilum ATCC 15354.

Examples of microorganisms belonging to the genus Brevibacterium include Brevibacterium divaricatum (Corynebacterium glutamicum) ATCC 14020, Brevibacterium flavum (Corynebacterium glutamicum ATCC 14067) ATCC 13826, ATCC 14067, Brevibacterium immariophilum ATCC 14068, Brevibacterium lactofeimentum (Corynebacterium glutamicum ATCC 13869) ATCC 13665, ATCC 13869, Brevibacterium roseum ATCC 13825, Brevibacterium saccharolyticum ATCC 14066, Brevibacterium tiogenitalis ATCC 19240, Brevibacterium album ATCC 15111, and Brevibacterium cerinum ATCC 15112.

Examples of microorganisms belonging to the genus Corynebacterium include Corynebacterium ammoniagenes ATCC 6871, ATCC 6872, Corynebacterium glutamicum ATCC 13032, Corynebacterium glutamicum ATCC 14067, Corynebacterium acetoacidophilum ATCC 13870, Corynebacterium·acetoglutamicum ATCC 15806, Corynebacterium alkanolyticum ATCC 21511, Corynebacterium callunae ATCC 15991, Corynebacterium glutamicum ATCC 13020, ATCC 13032, ATCC 13060, Corynebacterium lilium ATCC 15990, Corynebacterium melassecola ATCC 17965, Corynebacterium thermoaminogenes AJ12340 (FERM BP-1539), and Corynebacterium herculis ATCC 13868.

Examples of microorganisms belonging to the genus Pseudomonas include Pseudomonas putida, Pseudomonas fluorescens, Pseudomonas brassicacearum, Pseudomonas fulva, and Pseudomonas sp. D-0110.

As a method for introducing an expression vector into the foregoing host cell, any method can be used as long as it introduces DNA into the host cell. Examples thereof include a method using calcium ions [Proc. Natl. Acad. Sci. USA, 69, 2110 (1972)], a protoplast method (Japanese Unexamined Patent Publication No. S63-248394), or a method described in Gene, 17, 107 (1982) or Molecular & General Genetics, 168, 111 (1979).

Transformation of microorganisms belonging to the genus Brevibacillus can be carried out, for example, by the method of Takahashi et al. (J. Bacteriol., 1983, 156: 1130-1134), the method of Takagi et al. (Agric. Biol. Chem., 1989, 53: 3099-3100), or the method of Okamoto et al. (Biosci. Biotechnol. Biochem., 1997, 61: 202-203).

Examples of the vector into which the nucleic acid according to the present invention is introduced (hereinafter, simply referred to as “vector”) include pBTrp2, pBTac1, and pBTac2 (all commercially available from Boehringer Mannheim GmbH), pKK233-2 (manufactured by Pharmacia Corporation), pSE280 (manufactured by Invitrogen Corporation), pGEMEX-1 (manufactured by Promega Corporation), pQE-8 (manufactured by QIAGEN Corporation), pKYP10 (Japanese Unexamined Patent Publication No. S58-110600), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(−) (manufactured by Stratagene Corporation), pTrs30 (constructed from Escherichia coli JM109/pTrS30 (FERM BP-5407)], pTrs32 [constructed from Escherichia coli All 09/pTrS32 (FERM BP-5408)], pGHA2 [constructed from Escherichia coli IGHA2 (FERM B-400), Japanese Unexamined Patent Publication No. S60-221091], pTerm2 (U.S. Pat. Nos. 4,686,191, 4,939,094, 5,160,735), pSupex, pUB110, pTP5, pC194, pEG400 [J. Bacteriol., 172, 2392 (1990)], pGEX (manufactured by Pharmacia Corporation), and pET systems (manufactured by Novagen, Inc.).

In the case where Escherichia coli is used as a host, pUC18, pBluescriptII, pSupex, pET22b, pCold, or the like can be mentioned as a suitable vector.

Specific examples of vectors suitable for microorganisms belonging to the genus Brevibacillus include pUB110 or pHY500 (Japanese Unexamined Patent Publication No. H2-31682), pNY700 (Japanese Unexamined Patent Publication No. H4-278091), pHY4831 (J. Bacteriol., 1987, 1239-1245), pNU200 (UDAKA Shigezou, Journal of the Agricultural Chemical Society of Japan, 1987, 61: 669-676), pNU100 (Appl. Microbiol. Biotechnol., 1989, 30: 75-80), pNU211 (J. Biochem., 1992, 112: 488-491), pNU211R2L5 (Japanese Unexamined Patent Publication No. H7-170984), pNH301 (Appl. Environ. Microbiol., 1992, 58: 525-531), pNH326, pNH400 (J. Bacteriol., 1995, 177: 745-749), and pHT210 (Japanese Unexamined Patent Publication No. H6-133782), pHT110R2L5 (Appl. Microbiol. Biotechnol., 1994, 42: 358-363), which are known as Bacillus subtilis vectors; and pNCO2 (Japanese Unexamined Patent Publication No. 2002-238569) which is a shuttle vector between Escherichia coli and a microorganism belonging to the genus Brevibacillus.

The promoter is not limited as long as it functions in a host cell. Examples thereof include promoters derived from Escherichia coli or phage such as a trp promoter (Ptrp), a lac promoter, a PL promoter, a PR promoter, and a T7 promoter. Also, promoters artificially designed and modified, such as a promoter (Ptrp×2) in which two Ptrp are connected in series, a tac promoter, a lacT7 promoter, and a let I promoter, can also be used.

It is preferable to use a plasmid in which the distance between the Shine-Dalgarno sequence, which is a ribosome binding sequence, and the initiation codon is adjusted to an appropriate distance (for example, 6 to 18 bases). In the expression vector according to the present invention, a transcription termination sequence is not always necessary for the expression of the nucleic acid according to the present invention, but it is preferable to arrange a transcription termination sequence immediately below a structural gene.

Examples of eukaryotic hosts include yeast, filamentous fungi (mold and the like), and insect cells.

Examples of the yeast include yeasts belonging to the genus Saccharomyces, Schizosaccharomyces, Kluyveromyces, Trichosporon, Schwanniomyces, Pichia, Candida, Yarrowia, Hansenula, and the like. More specific examples of the yeast include Saccharomyces cerevisiae, Schizosaccharomyces pombe, Kluyveromyces lactis, Kluyveromyces marxianus, Trichosporon pullulans, Schwanniomyces alluvius, Schwanniomyces occidentalis, Candida utilis, Pichia pastoris, Pichia angusta, Pichia methanolica, Pichia polymorpha, Pichia stipitis, Yarrowia lipolytica, and Hansenula polymorpha.

It is preferred that the expression vector in the case where yeast is used as a host cell usually include an origin of replication (in the case where amplification in a host is required), a selection marker for propagation of the vector in Escherichia coli, a promoter and a terminator for recombinant protein expression in yeast, and a selection marker for yeast.

In the case where the expression vector is a non-integrating vector, it is preferable to further include an autonomously replicating sequence (ARS). This makes it possible to improve the stability of the expression vectors in cells (Myers, A. M., et al. (1986) Gene 45: 299-310).

Examples of the vector in the case where yeast is used as a host include YEP13 (ATCC 37115), YEp24 (ATCC 37051), YCp50 (ATCC 37419), YIp, pHS19, pHS15, pA0804, pHIL3OI, pHIL-S1, pPIC9K, pPICZα, pGAPZα, and pPICZ B.

The promoter is not limited as long as it can be expressed in yeast. Examples of the promoter include a promoter of glycolytic genes such as hexose kinase, a PHO5 promoter, a PGK promoter, a GAP promoter, an ADH promoter, a gal 1 promoter, a gal 10 promoter, a heat shock polypeptide promoter, an MFα1 promoter, a CUP 1 promoter, a pGAP promoter, a pGCW14 promoter, an AOX1 promoter, and an MOX promoter.

As a method for introducing an expression vector into yeast, any method can be used as long as it introduces DNA into yeast. Examples thereof include an electroporation method (Methods Enzymol., 194, 182 (1990)), a spheroplast method (Proc. Natl. Acad. Sci., USA, 81, 4889 (1984)), a lithium acetate method (J. Bacteriol., 153, 163 (1983)), and a method described in Proc. Natl. Acad. Sci. USA, 75, 1929 (1978).

Examples of filamentous fungi include fungi belonging to the genus Acremonium, Aspergillus, Ustilago, Trichoderma, Neurospora, Fusarium, Humicola, Penicillium, Myceliophtora, Botryts, Magnaporthe, Mucor, Metarhizium, Monascus, Rhizopus, and Rhizomucor.

Specific examples of filamentous fungi include Acremonium alabamense, Acremonium cellulolyticus, Aspergillus aculeates, Aspergillus awamori, Aspergillus oryzae, Aspergillus sake, Aspergillus sojae, Aspergillus tubigensis, Aspergillus niger, Aspergillus nidulans, Aspergillus parasiticus, Aspergillus ficuum, Aspergillus phoenicus, Aspergillus foetidus, Aspergillus flavus, Aspergillus fumigatus, Aspergillus japonicus, Trichoderma viride, Trichoderma harzianum, Trichoderma reseei, Chrysosporium lucknowense, Thermoascus, Sporotrichum, Sporotrichum cellulophilum, Talaromyces, Thielavia terrestris, Thielavia, Neurospora crassa, Fusarium oxysporus, Fusarium graminearum, Fusarium venenatum, Humicola insolens, Penicillium chrysogenum, Penicillium camemberti, Penicillium canescens, Penicillium emersonii, Penicillium fimiculosum, Penicillium griseoroseum, Penicillium purpurogenum, Penicillium roqueforti, Myceliophthora thermophilum, Mucor ambiguus, Mucor circinelloides, Mucor fragilis, Mucor hiemalis, Mucor inaequisporus, Mucor oblongiellipticus, Mucor racemosus, Mucor recurvus, Mucor saturninus, Mucor subtilissmus, Ogataea polymorpha, Phanerochaete chrysosporium, Rhizomucor miehei, Rhizomucor pusillus, and Rhizopus arrhizus.

The promoter in the case where the host is a filamentous fungus may be any one of a gene related to a glycolytic system, a gene related to constitutive expression, an enzyme gene related to hydrolysis, and the like. Specific examples thereof include amyB, glaA, agdA, glaB, TEF1, xynF1 tannase gene, No. 8AN, gpdA, pgkA, enoA, melO, sodM, catA, and catB.

Introduction of the expression vector into filamentous fungi can be carried out by a conventionally known method. Examples thereof include the method of Cohen et al. (calcium chloride method) [Proc. Natl. Acad. Sci. USA, 69: 2110 (1972)], a protoplast method [Mal. Gen. Genet., 168:111 (1979)], a competent method [J. Mol. Biol., 56: 209 (1971)], and an electroporation method.

Insect cells include, for example, lepidopteran insect cells, more specifically insect cells derived from Spodoptera frugiperda such as Sf9 and Sf21, and insect cells derived from Trichoplusia ni such as High 5.

Examples of the vector in the case where an insect cell is used as a host include baculoviruses such as Autographa californica nuclear polyhedrosis virus which is a virus that infects insects belonging to the family Noctuidae (Baculovirus Expression Vectors, A Laboratory Manual, W.H. Freeman and Company, New York (1992)).

In the case where an insect cell is used as a host, a polypeptide can be expressed by the method described in, for example, Current Protocols in Molecular Biology, Baculovirus Expression Vectors, A Laboratory Manual, W.H. Freeman and Company, New York (1992), or Bio/Technology, 6, 47 (1988). That is, a recombinant gene transfer vector and a baculovirus are co-introduced into an insect cell to obtain a recombinant virus (expression vector) in an insect cell culture supernatant, and then the recombinant virus is further infected into an insect cell, whereby the polypeptide can be expressed. Examples of the gene transfer vector used in the above method include pVL1392, pVL1393, and pBlueBaclll (all manufactured by Invitorogen Corporation).

As a method for co-introducing a recombinant gene transfer vector and a baculovirus into an insect cell for constructing the recombinant virus, for example, a calcium phosphate method (Japanese Unexamined Patent Publication No. H2-227075), a lipofection method (Proc. Natl. Acad. Sci. USA, 84, 7413 (1987)), or the like can be mentioned.

The recombinant vector according to the present invention preferably further contains a selection marker gene for selecting a transformant. For example, in Escherichia coli, resistance genes for various drugs such as tetracycline, ampicillin, and kanamycin can be used as selection marker genes. A recessive selection marker capable of complementing a genetic mutation involved in auxotrophy can also be used. In yeast, a resistance gene for geneticin can be used as a selection marker gene, and a gene complementing a genetic mutation involved in auxotrophy, or a selection marker such as LEU2, URA3, TRP1, or H1S3 can also be used. Examples of the selection marker gene for filamentous fungi include a marker gene selected from the group consisting of niaD (Biosci. Biotechnol. Biochem., 59, 1795-1797 (1995)), argB (Enzyme Microbiol Technol, 6, 386-389, (1984)), sC (Gene, 84, 329-334, (1989)), ptrA (BiosciBiotechnol Biochem, 64, 1416-1421, (2000)), pyrG (BiochemBiophys Res Commun, 112, 284-289, (1983)), amdS (Gene, 26, 205-221, (1983)), aureobasidin resistance gene (Mol Gen Genet, 261, 290-296, (1999)), benomyl resistance gene (Proc Natl Acad Sci USA, 83, 4869-4873, (1986)) and hygromycin resistance gene (Gene, 57, 21-26, (1987)), and a leucine auxotrophy-complementing gene. Further, in the case where the host is an auxotrophic mutant strain, a wild-type gene complementing the auxotrophy can also be used as a selection marker gene.

The selection of the host transformed with the expression vector according to the present invention can be carried out by plaque hybridization and colony hybridization using a probe that selectively binds to the nucleic acid according to the present invention. As the probe, it is possible to use a probe obtained by modifying a partial DNA fragment amplified by a PCR method based on sequence information of the nucleic acid according to the present invention with a radioisotope or digoxigenin.

(Production of Modified Fibroin)

In the host transformed with the expression vector according to the present invention, the modified fibroin according to the present invention can be produced by expressing the nucleic acid according to the present invention. As for the expression method, secretory production, fusion protein expression, or the like, in addition to direct expression, can be carried out according to the method described in Molecular Cloning, 2nd edition. In the case where it is expressed by yeast, an animal cell, or an insect cell, a modified fibroin can be obtained as a polypeptide to which a sugar or sugar chain is added.

The modified fibroin according to the present invention can be produced, for example, by culturing a host transformed with the expression vector according to the present invention in a culture medium, producing and accumulating the modified fibroin according to the present invention in the culture medium, and then collecting the modified fibroin from the culture medium. The method for culturing the host according to the present invention in a culture medium can be carried out according to a method commonly used for culturing a host.

In the case where the host according to the present invention is a prokaryote such as Escherichia coli or a eukaryote such as yeast, any of a natural medium and a synthetic medium may be used as a culture medium of the host according to the present invention as long as it contains a carbon source, a nitrogen source, inorganic salts and the like which can be assimilated by the host and it is capable of efficiently culturing the host.

As the carbon source, any carbon source that can be assimilated by the host may be used. Examples of the carbon source that can be used include carbohydrates such as glucose, fructose, sucrose, and molasses, starch and starch hydrolyzates containing them, organic acids such as acetic acid and propionic acid, and alcohols such as ethanol and propanol.

Examples of the nitrogen source that can be used include ammonium salts of inorganic or organic acids such as ammonia, ammonium chloride, ammonium sulfate, ammonium acetate and ammonium phosphate, other nitrogen-containing compounds, peptone, meat extract, yeast extract, corn steep liquor, casein hydrolyzate, soybean cake and soybean cake hydrolyzate, various fermented microbial cells and digested products thereof.

Examples of the inorganic salt that can be used include potassium dihydrogen phosphate, dipotassium phosphate, magnesium phosphate, magnesium sulfate, sodium chloride, ferrous sulfate, manganese sulfate, copper sulfate, and calcium carbonate.

Culture of a prokaryote such as Escherichia coli or a eukaryote such as yeast can be carried out under aerobic conditions such as shaking culture or deep aeration stirring culture. The culture temperature is, for example, 15° C. to 40° C. The culture time is usually 16 hours to 7 days. It is preferable to maintain the pH of the culture medium during the culture at 3.0 to 9.0. The pH of the culture medium can be adjusted using an inorganic acid, an organic acid, an alkali solution, urea, calcium carbonate, ammonia, or the like.

In addition, antibiotics such as ampicillin and tetracycline may be added to the culture medium as necessary during the culture. In the case of culturing a microorganism transformed with an expression vector using an inducible promoter as a promoter, an inducer may be added to the medium as necessary. For example, in the case of culturing a microorganism transformed with an expression vector using a lac promoter, isopropyl-β-D-thiogalactopyranoside or the like is used, and in the case of culturing a microorganism transformed with an expression vector using a trp promoter, indole acrylic acid or the like may be added to the medium.

As a culture medium for insect cells, commonly used TNM-FH medium (manufactured by Pharmingen Inc.), Sf-900 II SFM medium (manufactured by Life Technologies Corporation), ExCell 400 and ExCell 405 (both manufactured by JRH Biosciences Inc.), Grace's Insect Medium (Nature, 195, 788 (1962)), and the like can be used.

Culture of insect cells can be carried out, for example, for a culture time of 1 to 5 days under conditions such as pH 6 to 7 of culture medium and culture temperature 25° C. to 30° C. In addition, an antibiotic such as gentamicin may be added to the culture medium as necessary during the culture.

In the case where the host is a plant cell, the transformed plant cell may be directly cultured, or it may be differentiated into a plant organ and then cultured. As the culture medium for culturing a plant cell, for example, commonly used Murashige and Skoog (MS) medium, White medium, or a medium in which a plant hormone such as auxin or cytokinin is added to these media can be used.

Culture of animal cells can be carried out, for example, for a culture time of 3 to 60 days under conditions such as pH 5 to 9 of the culture medium and culture temperature 20° C. to 40° C. In addition, an antibiotic such as kanamycin or hygromycin may be added to the medium as necessary during the culture.

As a method for producing a modified fibroin using a host transformed with the expression vector according to the present invention, there are a method for producing the modified fibroin in a host cell, a method for secreting the modified fibroin outside the host cell, and a method for producing the modified fibroin on the outer membrane of the host cell. Each of these methods can be selected by changing the host cell to be used and the structure of the modified fibroin to be produced.

For example, in the case where a modified fibroin is produced in the host cell or on the outer membrane of the host cell, the production method can be altered to actively secrete the modified fibroin outside the host cell according to the method of Paulson et al. (J. Biol. Chem., 264, 17619 (1989)), the method of Lowe et al. (Proc. Natl. Acad. Sci. USA, 86, 8227 (1989), Genes Develop., 4, 1288 (1990)), or the methods described in Japanese Unexamined Patent Publication No. H5-336963, International Publication No. WO94/23021, and the like. That is, the modified fibroin can be actively secreted outside the host cell by expressing the modified fibroin in a form in which a signal peptide is added to a polypeptide containing an active site of a modified fibroin using a gene recombination technique.

The modified fibroin produced by the host transformed with the expression vector according to the present invention can be isolated and purified by a method commonly used for protein isolation and purification. For example, in the case where the modified fibroin is expressed in a dissolved state in cells, the host cells are recovered by centrifugation after completion of the culture, suspended in an aqueous buffer solution, and then disrupted using an ultrasonicator, a French press, a Manton-Gaulin homogenizer, a Dyno-Mill, or the like to obtain a cell-free extract. From the supernatant obtained by centrifuging the cell-free extract, a purified preparation can be obtained by a method commonly used for protein isolation and purification, that is, a solvent extraction method, a salting-out method using ammonium sulfate or the like, a desalting method, a precipitation method using an organic solvent, an anion exchange chromatography method using a resin such as diethylaminoethyl (DEAE)-Sepharose or DIAION HPA-75 (manufactured by Mitsubishi Kasei Kogyo Kabushiki Kaisha), an cation exchange chromatography method using a resin such as S-Sepharose FF (Pharmacia Corporation), a hydrophobic chromatography method using a resin such as butyl sepharose or phenyl sepharose, a gel filtration method using a molecular sieve, an affinity chromatography method, a chromatofocusing method, an electrophoresis method such as isoelectric focusing or the like, alone or in combination thereof.

As the chromatography, column chromatography using phenyl-TOYOPEARL (available from Tosoh Corporation), DEAE-TOYOPEARL (available from Tosoh Corporation), and Sephadex G-150 (available from Pharmacia Biotech Inc.) is preferably used.

In the case where the modified fibroin is expressed by the formation of an insoluble matter in the cell, similarly, the host cells are recovered, disrupted and centrifuged to recover the insoluble matter of the modified fibroin as a precipitated fraction. The recovered insoluble matter of the modified fibroin can be solubilized with a protein denaturing agent. After this operation, a purified preparation of modified fibroin can be obtained by the same isolation and purification method as described above.

In the case where a modified fibroin or a derivative in which a sugar chain has been added to the modified fibroin is secreted extracellularly, the modified fibroin or the derivative thereof can be recovered from the culture supernatant. That is, a culture supernatant is obtained by treating the culture by a technique such as centrifugation, and a purified preparation can be obtained from the culture supernatant by using the same isolation and purification method as described above.

(Spinning)

The modified fibroin according to the present invention may be further subjected to spinning after production and purification as described above. The modified fibroin according to the present invention can be spun by a method commonly used for spinning fibroin. For example, a fiber formed from the modified fibroin according to the present invention can be obtained by spinning a spinning solution (dope solution) in which the modified fibroin according to the present invention is dissolved in a solvent.

The spinning solution is prepared by adding a solvent to the modified fibroin and adjusting it to a spinnable viscosity. The solvent may be any solvent as long as it can dissolve the modified fibroin. Examples of the solvent include hexafluoroisopropanol (HFIP), hexafluoroacetone (HFA), dimethyl sulfoxide (DMSO), N,N-dimethylformamide (DMF), formic acid, aqueous solutions containing urea, guanidine, sodium dodecyl sulfate (SDS), lithium bromide, calcium chloride, and lithium thiocyanate.

An inorganic salt may be added to the spinning solution, as necessary. The inorganic salt may be, for example, an inorganic salt consisting of a Lewis acid and a Lewis base shown below. Examples of the Lewis base include an oxo acid ion (nitrate ion, perchlorate ion, or the like), a metal oxo acid ion (permanganate ion or the like), a halide ion, a thiocyanate ion, and a cyanate ion. Examples of the Lewis acid include a metal ion such as an alkali metal ion or an alkaline earth metal ion, a polyatomic ion such as an ammonium ion, and a complex ion. Specific examples of the inorganic salt consisting of a Lewis acid and a Lewis base include lithium salts such as lithium chloride, lithium bromide, lithium iodide, lithium nitrate, lithium perchlorate, and lithium thiocyanate; calcium salts such as calcium chloride, calcium bromide, calcium iodide, calcium nitrate, calcium perchlorate, and calcium thiocyanate; iron salts such as iron chloride, iron bromide, iron iodide, iron nitrate, iron perchlorate, and iron thiocyanate; aluminum salts such as aluminum chloride, aluminum bromide, aluminum iodide, aluminum nitrate, aluminum perchlorate, and aluminum thiocyanate; potassium salts such as potassium chloride, potassium bromide, potassium iodide, potassium nitrate, potassium perchlorate, and potassium thiocyanate; sodium salts such as sodium chloride, sodium bromide, sodium iodide, sodium nitrate, sodium perchlorate, and sodium thiocyanate; zinc salts such as zinc chloride, zinc bromide, zinc iodide, zinc nitrate, zinc perchlorate, and zinc thiocyanate; magnesium salts such as magnesium chloride, magnesium bromide, magnesium iodide, magnesium nitrate, magnesium perchlorate, and magnesium thiocyanate; barium salts such as barium chloride, barium bromide, barium iodide, barium nitrate, barium perchlorate, and barium thiocyanate; and strontium salts such as strontium chloride, strontium bromide, strontium iodide, strontium nitrate, strontium perchlorate, and strontium thiocyanate.

The viscosity of the spinning solution may be appropriately set according to the spinning method, and it can be set to 100 to 15,000 centipoise (cP) at 35° C., for example. The viscosity of the spinning solution can be measured, for example, by using an “EMS viscometer” (trade name, manufactured by Kyoto Electronics Manufacturing Co., Ltd.).

The spinning method is not particularly limited as long as it is a method capable of spinning the modified fibroin according to the present invention, and examples thereof include dry spinning, melt spinning, and wet spinning. As a preferred spinning method, wet spinning can be mentioned.

In wet spinning, a solvent in which a modified fibroin is dissolved is extruded from a spinneret (nozzle) into a coagulation liquid (coagulation liquid tank), and the modified fibroin is solidified in the coagulation liquid, whereby it is possible to obtain an undrawn yarn in the form of a thread. The coagulation liquid may be a solution capable of desolvation, and examples thereof include lower alcohols having 1 to 5 carbon atoms such as methanol, ethanol and 2-propanol, and acetone. Water may be appropriately added to the coagulation liquid. The temperature of the coagulation liquid is preferably 0° C. to 30° C. In the case where a syringe pump having a nozzle with a diameter of 0.1 to 0.6 mm is used as a spinneret, the extrusion rate is preferably 0.2 to 6.0 ml/hr and more preferably 1.4 to 4.0 ml/hr per hole. The length of the coagulation liquid tank may be any length as long as desolvation can be carried out efficiently, and it is, for example, 200 to 500 mm. The take-off speed of the undrawn yarn may be, for example, 1 to 20 m/min and preferably 1 to 3 m/min. The residence time may be, for example, 0.01 to 3 minutes and preferably 0.05 to 0.15 minutes. Further, drawing (pre-drawing) may be carried out in the coagulation liquid. In order to suppress the evaporation of a lower alcohol, the coagulation liquid may be maintained at a low temperature and the yarn may be taken off in the state of an undrawn yarn. The coagulation liquid tank may be provided in multiple stages, and the drawing may be carried out in each stage or a specific stage, as necessary.

The undrawn yarn (or pre-drawn yarn) obtained by the above method can be made into a drawn yarn (fibroin fiber) through a drawing step. As a drawing method, wet heat drawing, dry heat drawing, and the like can be mentioned.

The wet heat drawing can be carried out in warm water, in a solution obtained by adding an organic solvent or the like to warm water, or during steam heating. The temperature may be, for example, 50° C. to 90° C. and preferably 75° C. to 85° C. In wet heat drawing, undrawn yarn (or pre-drawn yarn) can be drawn, for example, 1 to 10 times, preferably 2 to 8 times.

Dry heat drawing can be carried out using an electric tube furnace, a dry heat plate, or the like. The temperature may be, for example, 140° C. to 270° C. and preferably 160° C. to 230° C. In dry heat drawing, undrawn yarn (or pre-drawn yarn) can be drawn, for example, 0.5 to 8 times, preferably 1 to 4 times.

The wet heat drawing and the dry heat drawing may be carried out individually, or they may be carried out in multiple stages or in combination. That is, wet heat drawing and dry heat drawing can be carried out in an appropriate combination in such a manner that the first stage drawing is carried out by wet heat drawing and the second stage drawing is carried out by dry heat drawing, or the first stage drawing is carried out by wet heat drawing and the second stage drawing is carried out by wet heat drawing, and the third stage drawing is further carried out by dry heat drawing.

The final draw ratio in the drawing step is, for example, 5 to 20 times and preferably 6 to 11 times, with respect to the undrawn yarn (or pre-drawn yarn).

The modified fibroin according to the present invention may be chemically crosslinked between polypeptide molecules in a fibroin fiber after being drawn into the fibroin fiber. Examples of the functional group which can be crosslinked include an amino group, a carboxyl group, a thiol group, and a hydroxy group. For example, an amino group of a lysine side chain contained in a polypeptide can be crosslinked with a carboxyl group of a glutamic acid or aspartic acid side chain by an amide bond through dehydration condensation. The crosslinking may be carried out by a dehydration condensation reaction under vacuum heating or may be carried out by using a dehydrating condensation agent such as carbodiimide.

The crosslinking between polypeptide molecules may be carried out using a crosslinking agent such as carbodiimide or glutaraldehyde or may be carried out using an enzyme such as transglutaminase. The carbodiimide is a compound represented by General Formula: R₁N═C═NR₂ (where R₁ and R₂ each independently represent an organic group including an alkyl group or cycloalkyl group having 1 to 6 carbon atoms). Specific examples of the carbodiimide include 1-ethyl-3-(3-dimethylaminopropyl)carbodiimide hydrochloride (EDC), N,N′-dicyclohexylcarbodiimide (DCC), 1-cyclohexyl-3-(2-morpholinoethyl)carbodiimide, and diisopropyl carbodiimide (DIC). Among them, EDC and DIC are preferred since they have a high ability to form an amide bond between polypeptide molecules and facilitate a crosslinking reaction.

The crosslinking treatment is preferably carried out by applying a crosslinking agent to the fibroin fiber and crosslinking it by vacuum heating drying. As the crosslinking agent, a pure product may be applied to the fibroin fiber. Alternatively, the crosslinking agent may be added to the fibroin fiber by diluting a pure product with a lower alcohol having 1 to 5 carbon atoms and a buffer solution or the like to a concentration of 0.005 to 10% by mass. The crosslinking treatment is preferably carried out at a temperature of 20° C. to 45° C. for 3 to 42 hours. By the crosslinking treatment, higher stress (strength) can be imparted to the fibroin fiber.

[Product]

The fibroin fiber formed from the modified fibroin according to the present invention can be applied as a fiber or a yarn to a woven fabric, a knitted fabric, a combination thereof, a nonwoven fabric, or the like. Such a fibroin fiber can also be applied to high strength applications such as ropes, surgical sutures, flexible stops for electrical parts, and physiologically active materials for implantation (for example, artificial ligament and aortic band).

The modified fibroin according to the present invention can also be applied to filaments, films, foams, spheres, nanofibrils, hydrogels, resins and equivalents thereof, which can be produced in accordance with the method described in Japanese Unexamined Patent Publication No. 2009-505668, Japanese Unexamined Patent Publication No. 2009-505668, Japanese Patent No. 5678283, Japanese Patent No. 4638735, or the like.

### EXAMPLES

Hereinafter, the present invention will be described more specifically with respect to Examples. However, the present invention is not limited to the following Examples.

[(1) Synthesis of Nucleic Acid Encoding Modified Fibroin and Construction of Expression Vector]

Based on the nucleotide sequence and amino acid sequence of Nephila clavipes (GenBank Accession Number: P46804.1, GI: 1174415) which is naturally occurring fibroin, fibroins and modified fibroins having amino acid sequences set forth in SEQ ID NOs: 1 to 4 and 6 to 11 were designed. The amino acid sequence set forth in SEQ ID NO: 1 is a sequence obtained by deleting alanine residues of an amino acid sequence in which the alanine residues in the (A)ₙ motif of the naturally occurring fibroin are consecutive so that the number of consecutive alanine residues is 5; and the amino acid sequence (PRT313) set forth in SEQ ID NO: 6 is a sequence obtained by adding the amino acid sequence (tag sequence and hinge sequence) set forth in SEQ ID NO: 5 to the N-terminal of the amino acid sequence (Met-PRT313) set forth in SEQ ID NO: 1 (Comparative Examples 1 and 2). The amino acid sequence (Met-PRT399) set forth in SEQ ID NO: 2 is a sequence obtained by deleting the (A)ₙ motif ((A)₅) every other two positions from the N-terminal side to the C-terminal side from the amino acid sequence set forth in SEQ ID NO: 1, and inserting one [(A)ₙ motif-REP] before the C-terminal sequence; and the amino acid sequence (PRT399) set forth in SEQ ID NO: 7 is a sequence obtained by adding the amino acid sequence (tag sequence and hinge sequence) set forth in SEQ ID NO: 5 to the N-terminal of the amino acid sequence set forth in SEQ ID NO: 2 (Examples 1 and 4). The amino acid sequence (Met-PRT380) set forth in SEQ ID NO: 3 is a sequence obtained by substituting GQX for all GGX in REP of the amino acid sequence set forth in SEQ ID NO: 1; and the amino acid sequence (PRT380) set forth in SEQ ID NO: 8 is a sequence obtained by adding the amino acid sequence (tag sequence and hinge sequence) set forth in SEQ ID NO: 5 to the N-terminal of the amino acid sequence set forth in SEQ ID NO: 3 (Reference Examples 1 and 2). The amino acid sequence (Met-PRT410) set forth in SEQ ID NO: 4 is a sequence obtained by substituting GQX for all GGX in REP of the amino acid sequence set forth in SEQ ID NO: 2; and the amino acid sequence (PRT410) set forth in SEQ ID NO: 9 is a sequence obtained by adding the amino acid sequence (tag sequence and hinge sequence) set forth in SEQ ID NO: 5 to the N-terminal of the amino acid sequence set forth in SEQ ID NO: 4 (Examples 2 and 5). The amino acid sequence (Met-PRT468) set forth in SEQ ID NO: 10 is a sequence obtained by inserting two alanine residues at the C-terminal side of each (A)ₙ motif of the amino acid sequence set forth in SEQ ID NO: 4 and further substituting a part of glutamine (Q) residues with a serine (S) residue to delete a part of amino acids on the N-terminal side so as to be almost the same as the molecular weight of SEQ ID NO: 4; and the amino acid sequence (PRT468) set forth in SEQ ID NO: 11 is a sequence obtained by adding the amino acid sequence (tag sequence and hinge sequence) set forth in SEQ ID NO: 5 to the N-terminal of the amino acid sequence set forth in SEQ ID NO: 10 (Example 3).

Each of nucleic acids encoding proteins having amino acid sequences set forth in SEQ ID NOs: 6 to 9 and 11 in which a His tag sequence and a hinge sequence (SEQ ID NO: 5) have been added to the N-terminal of each designed amino acid sequence set forth in SEQ ID NOs: 1 to 4 and 10 was synthesized. In the nucleic acid, an NdeI site was added to the 5′ end and an EcoRI site was added downstream of the stop codon. These four kinds of nucleic acids were cloned into a cloning vector (pUC118). Thereafter, the same nucleic acid was cleaved by restriction enzyme treatment with NdeI and EcoRI, and then recombined into a protein expression vector pET-22b(+) to obtain an expression vector.

[(2) Expression of Protein]

Escherichia coli BLR(DE3) was transformed with a pET22b(+) expression vector including each of nucleic acids encoding proteins having the amino acid sequences set forth in SEQ ID NOs: 6 to 9 and 11. The transformed Escherichia coli was cultured in 2 mL of an LB medium containing ampicillin for 15 hours. The culture solution was added to 100 mL of a seed culture medium (Table 6) containing ampicillin so that the OD₆₀₀ was 0.005. The temperature of the culture solution was maintained at 30° C. and the flask culture was carried out (for about 15 hours) until the OD₆₀₀ reached 5, thereby obtaining a seed culture solution.

TABLE 6
Seed culture medium

| Reagents | Concentration (g/L) |
|---|---|
| Glucose | 5.0 |
| KH₂PO₄ | 4.0 |
| K₂HPO₄ | 9.3 |
| Yeast Extract | 6.0 |
| Ampicillin | 0.1 |

The seed culture solution was added to a jar fermenter to which 500 ml of a production medium (Table 7) had been added so that the OD₆₀₀ was 0.05. The culture was carried out while maintaining the culture solution temperature at 37° C. and keeping the pH constant at 6.9. Further, the dissolved oxygen concentration in the culture solution was maintained at 20% of the dissolved oxygen saturation concentration.

TABLE 7
Production medium

| Reagents | Concentration (g/L) |
|---|---|
| Glucose | 12.0 |
| KH₂PO₄ | 9.0 |
| MgSO₄•7H₂O | 2.4 |
| Yeast Extract | 15 |
| FeSO₄•7H₂O | 0.04 |
| MnSO₄•5H₂O | 0.04 |
| CaCl₂•2H₂O | 0.04 |
| ADEKANOL | |
| (LG-295S, Adeka | 0.1 (mL/L) |
| Corporation) | |

Immediately after glucose in the production medium was completely consumed, a feed solution (455 g/1 L of glucose and 120 g/1 L of Yeast Extract) was added at a rate of 1 ml/min. The culture was carried out while maintaining the culture solution temperature at 37° C. and keeping the pH constant at 6.9. Further, the dissolved oxygen concentration in the culture solution was maintained at 20% of the dissolved oxygen saturation concentration, and the culture was carried out for 20 hours. Thereafter, 1 M isopropyl-β-thiogalactopyranoside (IPTG) was added to the culture solution to a final concentration of 1 mM to induce the expression of the target protein. Twenty hours after addition of IPTG, the culture solution was centrifuged to recover the bacterial cells. SDS-PAGE was carried out using the bacterial cells prepared from the culture solution before the addition of IPTG and after the addition of IPTG, and the expression of the target protein was confirmed by the appearance of a Band of a Target Protein Size Depending on the Addition of IPTG.

[(3) Purification of Protein]

The bacterial cells recovered 2 hours after the addition of IPTG were washed with 20 mM Tris-HCl buffer solution (pH 7.4). The bacterial cells after washing were suspended in 20 mM Tris-HCl buffer solution (pH 7.4) containing about 1 mM PMSF, and the cells were disrupted with a high-pressure homogenizer (available from GEA Niro Soavi SpA). The disrupted cells were centrifuged to obtain a precipitate. The obtained precipitate was washed with 20 mM Tris-HCl buffer solution (pH 7.4) until high purity. The precipitate after washing was suspended in 8 M guanidine buffer solution (8 M guanidine hydrochloride, 10 mM sodium dihydrogen phosphate, 20 mM NaCl, 1 mM Tris-HCl, pH 7.0) so as to have a concentration of 100 mg/mL, and dissolved by stirring with a stirrer at 60° C. for 30 minutes. After dissolution, dialysis was carried out with water using a dialysis tube (cellulose tube 36/32 manufactured by Sanko Junyaku Co., Ltd.). The white aggregated protein obtained after dialysis was recovered by centrifugation, the water content was removed with a freeze dryer, and the freeze-dried powder was recovered.

The degree of purification of the target protein in the freeze-dried powder thus obtained was confirmed by image analysis of polyacrylamide gel electrophoresis results of the powder using TotalLab (Nonlinear Dynamics Ltd.). As a result, the purity of each protein was about 85%.

[(4) Preparation of Spinning Solution (Dope Solution)]

Using DMSO in which 4% by mass of lithium chloride as an additive was previously dissolved as a main solvent, each freeze-dried powder of PRT313 (SEQ ID NO: 6: Comparative Example 1), PRT399 (SEQ ID NO: 7: Example 1), PRT380 (SEQ ID NO: 8: Reference Example 1), PRT410 (SEQ ID NO: 9: Example 2) and PRT468 (SEQ ID NO: 11: Example 3) proteins as prepared above was added to the main solvent to a concentration of 24% by mass. The freeze-dried powder was dissolved in a rotator at 90° for 1 hour and at 80° C. for 15 hours and then filtered through a sintered metal filter to remove dust. Subsequently, the filtrate was allowed to stand for 1 hour to remove foam to thereby prepare a spinning solution (dope solution). Although the viscosity of the spinning solution varies somewhat depending on the protein type and temperature, in the case of PRT410, it was 5,000 cP (centipoise) at 35° C.

[(5) Spinning]

The spinning solution was filled in a reserve tank and discharged from a multihole nozzle having a diameter of 0.1 or 0.2 mm into a 100% by mass methanol coagulation bath using a gear pump. The discharge amount was adjusted to 3 to 6 ml/min. After coagulation, washing and drawing were carried out in a 100% by mass methanol washing bath. After washing and drawing, it was dried using a dry hot plate and the obtained original yarn (fiber) was wound up.

[Measurement of Physical Properties]

Physical properties of the obtained original yarn were measured as follows.

(A) Fiber diameter was determined using an optical microscope.

(B) The stress, initial elastic modulus, and elongation (displacement at breakage, displacement) of the fiber were measured at a temperature of 20° C. and a relative humidity of 65% using a tensile tester (INSTRON 3342), and the toughness was calculated by the following formula. In the tensile test, it was measured at intervals of 10 ms. Each sample was adhered to a mold made of cardboard, the distance between the clamps was 20 mm, and the pulling speed was 10 mm/min. The load cell capacity was 10 N, and the clamping jig was clip type. The measured value was the average value of the number of samples n=5.

Toughness was calculated by the following calculation formula.

Toughness=[E/(r²×π×L)×1000](unit: MJ/m³)

in which

E: Fracture energy (unit: J)

r: Radius of fiber (unit: mm)

πPi

L: Distance between the clamps at the time of tensile test measurement: 20 mm

The amount of production of the frozen powder of each protein, and the stress, toughness and elongation of each original yarn were measured, and the results are shown in Table 8 as relative values in the case where the value of PRT313 (SEQ ID NO: 6: Comparative Example 1) is 100.

TABLE 8

| | | Amount of | | | |
|---|---|---|---|---|---|
| | | production | | Tough- | Elon- |
| | Desig- | of powder | Stress | ness | gation |
| | nation | (%) | (%) | (%) | (%) |
| Comparative | PRT313 | 100 | 100 | 100 | 100 |
| Example 1 | | | | | |
| Example 1 | PRT399 | 297 | — | — | — |
| Reference | PRT380 | 469 | — | — | — |
| Example 1 | | | | | |
| Example 2 | PRT410 | 579 | 84 | 108 | 131 |
| Example 3 | PRT468 | 762 | 69 | 113 | 164 |

Modified fibroin with a reduced content of (A)ₙ motif exhibited significantly improved productivity (Example 1). The modified fibroin with a reduced content of glycine residues in REP, in addition to having a reduced content of (A)ₙ motif, exhibited more significantly improved productivity and improved toughness and elongation (Examples 2 and 3).

Next, the spinning conditions were changed as shown below, and the purified proteins PRT313 (SEQ ID NO: 6: Comparative Example 2), PRT399 (SEQ ID NO: 7: Example 4), PRT380 (SEQ ID NO: 8: Reference Example 2) and PRT410 (SEQ ID NO: 9: Example 5) as prepared above were subjected to spinning. Physical properties of the proteins were measured and compared in the same manner as described above.

The spinning solution was prepared in the same manner as in the foregoing section “(4) Preparation of spinning solution (dope solution)”. The prepared spinning solution was filled in a reserve tank and discharged from a nozzle with a diameter of 0.2 mm into a 100% by mass methanol coagulation bath using a gear pump. The discharge amount was adjusted to 0.050 to 0.052 ml/min. After coagulation, washing was carried out in a 100% by mass methanol washing bath, and 3-fold drawing was carried out in a hot water bath at 50° C. After washing and drawing, it was dried using a hot roller at 60° C., and the obtained original yarn (fiber) was wound up.

The measurement results of the stress, toughness and elongation of each original yarn are shown in Table 9 as relative values in the case where the value of PRT313 (SEQ ID NO: 6: Comparative Example 2) is 100.

TABLE 9

| | | Stress | Toughness | Elongation |
|---|---|---|---|---|
| | Designation | (%) | (%) | (%) |
| Comparative | PRT313 | 100.0 | 100.0 | 100.0 |
| Example 2 | | | | |
| Example 4 | PRT399 | 102.5 | 131.5 | 152.8 |
| Reference | PRT380 | 99.8 | 89.0 | 94.4 |
| Example 2 | | | | |
| Example 5 | PRT410 | 92.2 | 125.1 | 168.4 |

Modified fibroin with a reduced (A)ₙ motif content exhibited improved productivity while maintaining stress, and simultaneously also exhibited improved toughness and elongation (Example 4). Modified fibroin with a reduced content of glycine residues in REP, in addition to having a reduced content of (A)ₙ motif, also exhibited the same results (Example 5).

1. A modified fibroin, comprising:
a domain sequence represented by Formula 1: [(A)ₙ motif-REP]ₘ,
wherein the domain sequence has an amino acid sequence having a reduced content of (A)ₙ motif equivalent to an amino acid sequence in which, at least, one or a plurality of the (A)ₙ motifs is deleted, as compared to naturally occurring fibroin;
wherein in Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.

2. The modified fibroin according to claim 1, wherein the domain sequence has an amino acid sequence equivalent to an amino acid sequence in which, at least, one (A)ₙ motif per one to three (A)ₙ motifs from the N-terminal side to the C-terminal side is deleted, as compared to the naturally occurring fibroin.

3. The modified fibroin according to claim 1, wherein the domain sequence has an amino acid sequence equivalent to an amino acid sequence in which, at least, two consecutive (A)ₙ motif deletions and one (A)ₙ motif deletion are repeated in this order from the N-terminal side to the C-terminal side, as compared to the naturally occurring fibroin.

4. A modified fibroin, comprising:
a domain sequence represented by Formula 1: [(A)ₙ motif-REP]ₘ,
wherein x/y is 50% or more, in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, a maximum value of the total value of the number of amino acid residues in the two adjacent [(A)ₙ motif-REP] units where the ratio of the number of amino acid residues in the other REP is 1.8 to 11.3 is defined as x, and the total number of amino acid residues of the domain sequence is defined as y;
wherein in Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.

5. The modified fibroin according to claim 1, wherein the fibroin has, in addition to an amino acid sequence corresponding to deletion of one or a plurality of (A)ₙ motifs, an amino acid sequence corresponding to substitution, deletion, insertion and/or addition of one or a plurality of amino acid residues, as compared to naturally occurring fibroin.

6. The modified fibroin according to claim 5, wherein the naturally occurring fibroin is a fibroin derived from an insect or a spider.

7. The modified fibroin according to claim 5, wherein the naturally occurring fibroin is a major ampullate spider protein (MaSp) or minor ampullate spider protein (MiSp) of spiders.

8. The modified fibroin according to claim 5, wherein the domain sequence has an amino acid sequence equivalent to an amino acid sequence in which, at least, one (A)ₙ motif per one to three (A)ₙ motifs from the N-terminal side to the C-terminal side is deleted, as compared to the naturally occurring fibroin.

9. The modified fibroin according to claim 5, wherein the domain sequence has an amino acid sequence equivalent to an amino acid sequence in which, at least, two consecutive (A)ₙ motif deletions and one (A)ₙ motif deletion are repeated in this order from the N-terminal side to the C-terminal side, as compared to the naturally occurring fibroin.

10. The modified fibroin according to claim 1, wherein the domain sequence has an amino acid sequence having a reduced content of glycine residues equivalent to an amino acid sequence in which, at least, one or a plurality of the glycine residues in REP is substituted with another amino acid residue, as compared to the naturally occurring fibroin.

11. The modified fibroin according to claim 10, wherein the domain sequence has an amino acid sequence equivalent to an amino acid sequence in which, at least, in at least one motif sequence selected from GGX and GPGXX (where X represents an amino acid residue other than glycine) in REP, one glycine residue in one or a plurality of the motif sequences is substituted with another amino acid residue, as compared to the naturally occurring fibroin.

12. The modified fibroin according to claim 11, wherein the ratio of the motif sequence having the substitution of a glycine residue with another amino acid residue is 40% or more with respect to the entire motif sequence.

13. The modified fibroin according to claim 10, wherein z/w is 30% or more in the case where the total number of amino acid residues in the amino acid sequence consisting of XGX (where X represents an amino acid residue other than glycine) contained in all REPs in the sequence excluding the sequence from the (A)ₙ motif located at the most C-terminal side to the C-terminal of the domain sequence from the domain sequence is defined as z, and the total number of amino acid residues in the sequence excluding the sequence from the (A)ₙ motif located at the most C-terminal side to the C-terminal of the domain sequence from the domain sequence is defined as w.

14. A modified fibroin, comprising an amino acid sequence set forth in SEQ ID NO: 2, SEQ ID NO: 4 or SEQ ID NO: 10, or an amino acid sequence having 90% or more sequence identity with the amino acid sequence set forth in SEQ ID NO: 2, SEQ ID NO: 4 or SEQ ID NO: 10.

15. The modified fibroin according to claim 1, further comprising a tag sequence at either or both of the N-terminal and the C-terminal.

16. The modified fibroin according to claim 15, wherein the tag sequence includes an amino acid sequence set forth in SEQ ID NO: 5.

17. A modified fibroin, comprising an amino acid sequence set forth in SEQ ID NO: 7, SEQ ID NO: 9 or SEQ ID NO: 11, or an amino acid sequence having 90% or more sequence identity with the amino acid sequence set forth in SEQ ID NO: 7, SEQ ID NO: 9 or SEQ ID NO: 11.

18. A nucleic acid encoding the modified fibroin according to claim 1.

19. A nucleic acid that hybridizes with a complementary strand of the nucleic acid according to claim 18 under stringent conditions and encodes a modified fibroin including a domain sequence represented by Formula 1: [(A)ₙ motif-REP]ₘ;
wherein in Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.

20. A nucleic acid having 90% or more sequence identity with the nucleic acid according to claim 18 and encoding a modified fibroin including a domain sequence represented by Formula 1: [(A)ₙ motif-REP]ₘ;
wherein in Formula 1, (A)ₙ motif represents an amino acid sequence consisting of 4 to 20 amino acid residues and the number of alanine residues relative to the total number of amino acid residues in the (A)ₙ motif is 83% or more, REP represents an amino acid sequence consisting of 10 to 200 amino acid residues, m represents an integer of 8 to 300, a plurality of (A)ₙ motifs may be the same amino acid sequence or different amino acid sequences, and a plurality of REPs may be the same amino acid sequence or different amino acid sequences.

21. An expression vector, comprising the nucleic acid sequence according to claim 18; and one or a plurality of regulatory sequences operably linked thereto.

22. (canceled)

23. A host transformed with the expression vector according to claim 21.

24-33. (canceled)

34. A product comprising the modified fibroin according to claim 1 and selected from the group consisting of a fiber, a yarn, a filament, a film, a foam, a sphere, a nanofibril, a hydrogel, a resin and an equivalent thereof.


SEQUENCE LISTING (11 sequences)

SEQ ID NO: 1 - protein, 597 aa, Artificial Sequence
  Met-PRT313 Synthetic Sequence
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 2 - protein, 590 aa, Artificial Sequence
  Met-PRT399 Synthetic Sequence
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 3 - protein, 597 aa, Artificial Sequence
  Met-PRT380 Synthetic Sequence
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 4 - protein, 590 aa, Artificial Sequence
  Met-PRT410 Synthetic Sequence
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 5 - protein, 12 aa, Artificial Sequence
  HisTag Synthetic Sequence
MHHHHHHSSGSS

SEQ ID NO: 6 - protein, 608 aa, Artificial Sequence
  PRT313 Synthetic Sequence
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 7 - protein, 601 aa, Artificial Sequence
  PRT399 Synthetic Sequence
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 8 - protein, 608 aa, Artificial Sequence
  PRT380 Synthetic Sequence
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 9 - protein, 601 aa, Artificial Sequence
  PRT410 Synthetic Sequence
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 10 - protein, 565 aa, Artificial Sequence
  Met-PRT468 Synthetic Sequence
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 11 - protein, 576 aa, Artificial Sequence
  PRT468 Synthetic Sequence
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS